(19) United States
(12) Patent Application Publication
(10) Pub. No.: US 2024/0415640 A1
(43) Pub. Date: Dec. 19, 2024

(54) Aortic Heart Valve Devices

(71) Applicants: Raghuveer Basude, Newark, CA (US); Aishwarya Basude, Newark, CA (US); Shri Krishna Basude, Newark, CA (US)

(72) Inventors: Raghuveer Basude, Newark, CA (US); Aishwarya Basude, Newark, CA (US); Shri Krishna Basude, Newark, CA (US)

(73) Assignee: Dr. Raghuveer Basude, Newark, CA (US)

(21) Appl. No.: 18/668,242

(22) Filed: May 19, 2024

Related U.S. Application Data
(60) Provisional application No. 63/562,978, filed on Mar. 8, 2024.
(60) Provisional application No. 63/507,736, filed on Jun. 13, 2023.
(60) Provisional application No. 63/591,052, filed on Oct. 17, 2023.
(60) Provisional application No. 63/519,821, filed on Aug. 15, 2023.
(60) Provisional application No. 63/509,664, filed on Jun. 22, 2023.
(60) Provisional application No. 63/503,437, filed on May 19, 2023.

(51) Int. Cl. A61F 2/24

(52) U.S. Cl. CPC A61F 2/2418; A61F 2250/0059

(57) ABSTRACT

The present disclosure relates generally to the systems, devices and methods for devices, and systems to treat aortic heart valve. The patent describes prosthetic valve embodiments with actuatable arms, which grasp onto leaflets and significantly reduce risk of device migration and device size. The patent describes methods and embodiment to cut the diseased leaflets prior to prosthetic valve implantation, optionally under optical visualization. The patent describes prosthetic valve devices with reduced bare metal, valve housing units, and replaceable prosthetic leaflets. The patent describes atraumatic annular repair method and embodiments.

### CROSS-REFERENCE TO RELATED APPLICATIONS

This application claims the benefit of provisional patent application No. 63/503,437, filed on May 19, 2023; 63/507,736, filed on Jun. 13, 2023; the full disclosure of which are incorporated herein by reference.

### FIELD OF THE INVENTION

The present invention relates generally to medical methods, devices, and systems to treat aortic heart valve. In particular, the present invention relates to methods, devices, and systems for the endovascular, percutaneous, or minimally invasive surgical treatment of bodily tissues, such as tissue approximation or valve repair/replacement. More particularly, the present invention relates to methods and devices for the repair or replacement of aortic valve, however, these novelties may be applied to treat pulmonary, mitral, and tricuspid heart valves, venous valves, and other tissue structure through minimally invasive and other procedures, as obvious to Person of Ordinary Skill in the Art (POSA).

### BACKGROUND OF THE INVENTION

### Heart Valve Devices:

Atraumatic valves: Most prosthetic devices used for heart valve replacement, such as those designed for the aortic valve, are held in place by compressing against the anulus or the wall to mitigate perivalvular leaks and device migration. To achieve this, these devices need to be highly rigid or apply significant expansion force, often necessitating larger dimensions in terms of both length and diameter to minimize the risk of migration. However, the increased size, stiffness, and length of these devices can negatively impact the surrounding anatomy and blood interactions, potentially leading to issues like heightened stress points, deformations, restricted blood flow (ischemia), tissue death (necrosis), and other complications.

Actuatable arms for secure positioning: Alternative devices, like the Jenavalve are capable of engaging with the leaflet, primarily for positioning purposes. Other examples, such as and U.S. Pat. No. 10,925,724 B2 engage with the leaflets, however, in order to engage or disengage with the leaflets, manipulation of prosthetic valve is required. That is, in all of these devices, the leaflet capture elements lack the ability to be independently actuated (for example, without disturbing the main body of the prosthetic valve) to securely grasp or ungrasp the leaflets.

Spacer and or flap devices: In some cases, there is regurgitation in aortic valve, as there is a gap between the leaflets. This can happen due to stiffening, tears, and other disease conditions. In these cases, there is a need for a simple spacer or flap devices that can effectively and functionally seal the regurgitation. This solution/treatment can be a temporary, semi-permanent, or permanent solution. Temporary or semi-permanent solutions are typically a bridge to a more permanent solution, such as total valve replacement.

Leaflet replacement: The typical lifespan of most prosthetic tissue valves ranges from 5 to 15 years. The current advanced transcatheter procedure involves deploying a new prosthetic valve within the failed tissue valve. However, the limited space between the calcified native valve and the failed prosthetic valve poses a challenge, resulting in restricted blood flow upon the insertion of a second valve. Consequently, there arises a necessity to either trim the leaflets of the failed prosthetic valve or completely remove the failed prosthetic valve in order to create adequate space for the new prosthetic valve to function effectively.

Leaflet shape and attachment: Native leaflets of the aortic valve are attached at the base of annulus and seat below or at the annulus. However, in current prosthetic heart valves, the prosthetic leaflets are supported along the height of the prosthetic valve frame and seat above the annulus. This results in hemodynamics flow patterns and stresses that are different from natural physiological flow patterns. Hence, there is a need to better mimic the natural flow patterns, by developing prosthetic valves that better resemble natural leaflet structures.

Mechanical valves boast a lengthy lifespan of over 20 years but necessitate ongoing blood thinner usage. Therefore, there's a pressing need to enhance the hemocompatibility of these mechanical valves. Additionally, there's a demand for the transcatheter placement of mechanical valves to further improve their accessibility and application in medical procedures.

Atraumatic annulus augmentation: The current state of the art transcatheter treatment for mitral valve regurgitation is edge-to-edge repair. However, there's potential for enhanced outcomes through a viable transcatheter annular reduction approach. Present solutions for annular reduction involve the insertion of multiple screws/anchors into the annulus, which not only heightens the risk of injury but also complicates the procedure excessively. Moreover, conducting annular reduction during the procedure can lead to tearing or tissue trauma by the screws/anchors, escalating the risk of arrhythmias and making it an impractical solution. Therefore, there's a necessity for an atraumatic transcatheter annular repair device. Additionally, it would be preferable if this device could be adjusted post-implantation or auto cinch to optimize its effectiveness by encouraging reverse remodeling the heart.

### SUMMARY OF THE INVENTION

The majority of prosthetic devices used for valve replacement, such as those for the aortic valve, rely on compression against the anulus or the wall, demanding high stiffness or substantial expansion force. Additionally, these devices often need to be longer than necessary to prevent migration or placement errors. An advantageous aspect of this innovation lies in its ability to grasp the leaflets, effectively reducing the risk of device migration and allowing for a smaller device size to secure it. Furthermore, this invention teaches methods and embodiments that allow for cutting diseased native and/or prosthetic leaflets, including complete removal of prosthetic valve. It also teaches modular or insertable/replaceable prosthetic leaflets within a valve. This invention also teaches means to enable blood flow to the coronary arteries during the valve replacement procedure, especially if the diseased leaflets are cut/removed. This invention also teaches methods and embodiments for optical visualization during the valve replacement procedure.

Conventional prosthetic mechanical valves typically feature exposed bare metal that isn't intended to be covered by tissue, thus requiring ongoing use of blood thinners to prevent clotting. One key advantage of this invention is its minimal or absent exposed bare metal surfaces that come into contact with blood flow. Furthermore, this invention teaches mechanical valves with minimum or no moving metallic parts. Additionally, this invention teaches methods and embodiments to deliver and implant using a catheter.

This invention also teaches annulus repair of mitral or tricuspid valve. This invention also teaches to atraumatically and securely attach the device to the annular tissue. This invention also teaches to cinch the annulus acutely (during the procedure) and also chronically (post procedure).

While the exemplary embodiments in this innovation primarily focus on the aortic valve, the design concepts can be extended to encompass all heart valves (pulmonary, mitral, tricuspid, aortic), pulmonary valves, GI valves, and other applications evident to those skilled in the field.

The following numbered clauses describe other examples, aspects, and embodiments of inventions described herein:
1. The method of deploying the prosthetic valve with actuating arms to secure against the native valve wall, annulus, or leaflets.
2. A device comprising a prosthetic valve with actuatable arms which can be sequentially or simultaneously actuated to grasp the native leaflets reversibly and repeatedly, while the prosthetic valve is fully expanded state.
3. The method of deploying the prosthetic valve in clause 1 with optical visualization using a camera, in addition to other modes of visualization.
4. The deployment system in clause 1 wherein the leaflets or the entirety of a failed prosthetic valve may be cut using a cutter to make room for new valve in valve prosthetic device.
5. An aortic prosthetic device with leaflet grasping arms attached comprising an elastic such as nitinol, metal, plastic, ceramic or their combination and a lever arm used for mechanical advantage that can be attached to prosthetic valve using a feature such as a suture, a hinge, weld, glue, screw, or rivet. The arms can have barbs or other frictional elements to securely grasp the leaflets. The device can be repositioned, redeployed, or removed, by actuating the arms, if necessary.
6. The deployment system in clause 1 wherein diseased and stenosed native leaflets may be cut to make room for new valve in valve prosthetic device.
7. A replaceable prosthetic leaflet device comprising of valve housing with fixation features such as a frame, arms, and a skirt, remaining fixed to the surrounding tissue post implantation, and a replaceable valve described in clause 5.
8. The aortic prosthetic device in clause 5 wherein the prosthetic valve can be repositioned, deployed and assessed multiple times in its fully expanded state.
9. The aortic prosthetic device in clause 5 wherein the arms are multiple components configured to be biased towards each other attached at their base or anywhere between the arm and the device, allowing for the arms to be raised or rotated with components such as a suture, screw mechanism, hydraulic, electrical, chemical, pneumatic etc.

The replaceable prosthetic leaflet design in clause 7 wherein the skirt is a hollow, balloon, stent with a cover, or any structure with a cover, an expandable and compressible structure, perforated structure, fabric, braid, film, coating, cushion, sponge, sponge like structure, or any structure that can prevent blood leaks.
8. The replaceable prosthetic leaflet design in clause 7 wherein one or more retrievable features such as suture loop, a hook, magnet, threads, or any commonly known features that aid retrieval of device post implantation (see co-owned patent for additional details). The retrievable features can also be actuatable, deployable, or remotely triggerable.
9. A prosthetic valve with retrievable features such as those in clause 8 wherein they optionally have features that aid in detection, for example radio-opaque, echogenic, magnetic, florescent, etc. The features can be of polymer, metal, ceramic, nitinol, etc.
10. The replaceable prosthetic leaflet design in clause 7 with wherein retrievable features may be placed both on the replaceable valve and valve housing so the features on the replaceable valve may be used to detach, remove, and replace the valve housing which may have stabilizers such as the studs, hooks, or slots.
11. The replaceable prosthetic leaflet design in clause 7 with tearable valve leaflets and a secondary frame, to easily remove it. Once removed the new valve can fit inside of it.
12. The replaceable prosthetic leaflet design in clause 7 wherein the replaceable valve has a retrievable feature to pull the failed leaflets out of the slot of the valve housing. The new replaceable valve can then be press fit inside the valve housing by using a balloon. Once secure, the balloon is then removed.
13. The replaceable prosthetic leaflet design in clause 7 wherein the replaceable valve has a click, elastic, or super elastic fit.
14. The replaceable prosthetic leaflet design in clause 7 wherein the replaceable valve has a twist fit.
15. The replaceable prosthetic leaflet design in clause 7 wherein the replaceable valve has a screw fit.
16. The replaceable prosthetic leaflet design in clause 7 wherein the replaceable valve can reversibly be secured or removed using ultrasonic, laser, thermal, UV, glue, friction, conical/pin fit, and/or mechanical fit.
17. The replaceable prosthetic leaflet design in clause 7 wherein a spacer is used to mitigate regurgitation. The shape and cross-section can be any geometric shape, including a cylindrical, star, triangular, flat, crescent, ellipsoidal, etc. Further, the spacer can be expandable, compressible, stretchable, inflatable, porous, solid, wire form, or sponge.
18. The device with the spacer in clause 17 such that it is smart, and the shape may be changed remotely electronically or hydraulically, or mechanically or chemically but not limited to these examples.
19. The replaceable prosthetic leaflet design in clause 7 wherein a tarp, leaflet, or disc is used to mitigate regurgitation. The tarp can be flexible, rigid, stretchable, non-stretchable, hollow, balloon, stent with a cover, or any structure with a cover, an expandable and compressible structure, perforated structure, fabric, braid, film, mesh, fibrous, coated, cushion, sponge, sponge like structure, or any structure that can mitigate valve regurgitation when placed over the gap between the leaflets. The tarp can be made of metal, plastic, biological origin, non-biological origin, ceramic, superelastic, shape memory, composite, fabric, braid, machined, 3d printed, or any combination known to POSA. The tarp can comprise of at least one structure, 2 structures or 3 or more structures. It can have single leaflet covering, double leaflet covering, or 3 or more leaflet covering. It can be of any shape, circular, pizza, triangular, trapezoid, square, oval, and so on. It can be corrugated, folded, smooth, fan like bladed structure, Japanese foldable fan like structure, with slits or without any slits. The tarp can have features, stiffening members, wires, sheets, along the rim and sides to configure and optimize the shape during diastole (to minimize regurgitation) and during systole (to maximize blood flow).
20. The replaceable prosthetic leaflet design in clause 7 wherein the mechanical prosthetic valve includes a coating, fabric, or braid that promotes tissue encapsulation and endothelization of the leaflets and valve body, to mitigate the need for blood thinners.
21. The replaceable prosthetic leaflet design in clause 7 wherein the mechanical prosthetic valve includes rubber like soft seal, to mitigate the valve opening and closing, clicking noise. The soft seal is achieved with use of a polymer O-ring, seating, tissue growth, or fabric cushion.
23. The replaceable prosthetic leaflet design in clause 7 in which the mechanical prosthetic valve uses polymer leaflets instead of metallic leaflets, for example, with polypropylene of polyurethane leaflets.
24. The replaceable prosthetic leaflet design in clause 7 in which the prosthetic mechanical valve comprises of uni-structure leaflets that bend/flex to open or close, instead of a hinged and two or more leaflets in typical mechanical valve
25. The replaceable prosthetic leaflet design in clause 7 in which the prosthetic mechanical valve comprises of 3D printed composite leaflets designed to mitigate the need for blood thinners and clicking noise. The 3D printed composite leaflet will have a cushioned seating when closing.
26. The method of deploying the prosthetic valve in clause 1 wherein a temporary one-way-valve is placed upstream to the valve so that blood flow to the coronary arteries is not compromised if removal of a failed prosthetic valve or cutting of prosthetic leaflets is required.
27. The replaceable prosthetic leaflet design in clause 7 wherein a filter comprising of a mesh and a self sealing or sealable port to advance various catheters can be placed downstream of the coronary sinus, in which case, debris free blood to the coronaries can be provided using tubes. The seal of the blood flow to coronary may be achieved using commonly known means, including a snug fit tube, tapered tube, inflatable or spongy cuff, a stent, O-ring, etc.
28. The replaceable prosthetic leaflet design in clause 7 functions of filter and temporary valve can be combined into a single embodiment.
29. The method of using the temporary valve seen in clause 26 such that it is a two-way valve (flow in both directions). This can be achieved mechanically, electrically, electronically, chemically, magnetically, using software, fluidic trigger, etc.
30. The method of using the temporary valve seen in clause 26 such that a guidewire is put through the failed valve and a cuff which can be a balloon, hemispheric, concave, cup shaped, bowl shaped, expandable umbrella like structure, disc, stent, or any feature used to restrict blood flow is deployed.
30. The method of using the temporary valve and a cuff seen in clause 27 such that the cuff has a port that is used to flush the working zone around the failed valve with clear, translucent, or transparent liquid such as heparinized saline, to aid visualization using cameras or optical techniques.
31. A annular ring cinching device wherein a stent design cut from a tube is used and a typical compression spring of circular cross-section is fastened, welded, sutured, or interlaced. The ring can be passed through a compression spring and compressed by cinching the string. The ring may be made of a compression spring with typical circular profile, a stent with a compression spring, a flattened compression spring with an oval, rectangular, triangular, trapezoidal, c-shaped, s-shaped or any other suitable profile. The compressible springs can be made of wire, sheet, tube, or 3D printed. It can have typical stent, braided, woven, coiled, or any suitable means.
32. The annular ring cinching device in clause 31, such that ring can has barbs or other frictional elements that cause none or minimal trauma to the tissue.
33. The method of deploying the prosthetic valve in clause 1 with left ventricular assist device (LVAD) or heart pump, to off-load the pressure across the aortic heart valve during the procedure.
34. The method of annular repair, wherein, the device is atraumatically deployed around the annulus, and the device cinched at a later date post tissue encapsulation, to cinch the annulus to mitigate regurgitation.

### BRIEF DESCRIPTION OF DRAWINGS

FIG. 1 to FIG. 25 primarily focus on aortic valve devices and methods.

FIG. 26 to FIG. 30 primarily focus on mitral valve annular repair.

The following is a listing of the reference numbers used in this application:

| LAA | Left Atrial Appendage |
|---|---|
| LA | Left Atrium |
| ORI | Orifice, Orifice or ostia of the LAA |
| D | Depth or Distance of the proximal face of the closure device |
| | behind the ORI (inside LAA) |
| AV | Aortic Valve |
| AA | Aortic Arch |
| DA | Descending Aorta |
| A | Aorta |
| LF | Augmenting Leaflets |
| CA | Coronary artery |
| CO | Coronary ostia |
| LF | Leaflets |
| LV | Left Ventricle |
| AC | Aortic Canal |
| LA | Left Atrium |
| CS | Coronary Sinus |
| CA | Coronary Artery |
| G | Gap |
| 1005, 1010 | Guidewire |
| 1012 | Catheter |
| 1016 | Lever arm, inverter |
| 1018 | Suture, hinge, weld, glue, screw, rivet |
| 1019 | Continuous segment of arm, for example, with a U-bend |
| 1020, 1022, 1024, | Grasping Arms (outer) |
| 1026 | |
| 1021, 1027 | Grasping Arms (inner) |
| 1032, 1034, 104, | Sutures |
| 1056 | |
| 1040 | Prosthetic Aortic Valve Device |
| 1042 | Valve Housing |
| 1046 | Lever arm |
| 1062 | Skirt |
| 1064 | Position markers |
| 1070 | Flexible tarp, film |
| 1074 | Support feature (with have barbs, clasps, crocodile teeth) |
| 1076 | Suture, wire, stent structure, tube |
| 1078 | Stent |
| 1080 | Spacer |
| 1090 | Lid |
| 1100 | Failed Prosthetic Valve |
| 1110 | Cutter Device |
| 1130, 1140 | Temporary Valve, |
| 1142 | One way valve |
| 1145 | Seal sealing or sealable port |
| 1148 | Mesh, filter |
| 1150 | Filter |
| 1155 | Balloon cuff |
| 1160 | Interfacing member |
| 1164 | Tubes |
| 1166 | O-Ring |
| 1170 | Catheter shaft |
| 1200 | Inflatable/deflatable Cuff |
| 1210, 1220, 1230, | Port/hole (optionally, the port hole can comprise of an LVAD |
| 1240 | motor/pump to off-load the pressure across the aortic valve) |
| 1250 | Camera |
| 1300, 1310, 1320 | Valve retrievable features (suture loop, a hook, magnet, threads) |
| 1330 | Replaceable Frame |
| 1405 | Frame (rigid, or stent like, compressible) |
| 1410 | Stationary rigid member, stationary telescoping member |
| 1420 | Trap |
| 1425 | Curved base |
| 1430 | Holes in the base |
| 1440 | Various designs of flow pattern and damping base structure to |
| | improve hemodynamics/hemocompatibility |
| 1500, 1510, | Ring |
| 1540 | Adjustable/retrieval feature |
| 1550 | Catheter |
| 1560 | Sutures, wires, tubes |
| 1570 | Torque cable |
| 1580 | Tube |
| 1585 | Balloon |

### DETAILED DESCRIPTION OF DRAWINGS

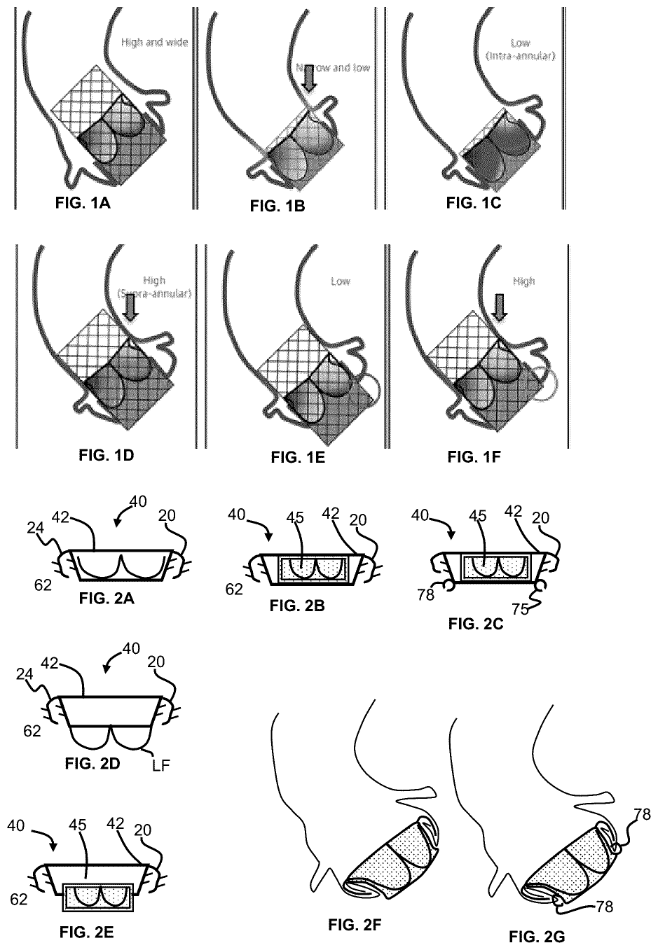

FIG. 1A shows an exemplary prior art with high frame aortic prosthetic valve that is correctly positioned, without blocking coronary artery ostia. FIG. 1B shows a short height frame aortic prosthetic valve that is not aligned well causing obstruction of the coronary artery ostia, while FIG. 1C shows correct placement. FIG. 1D shows incorrect placement or size of the prosthetic valve, causing coronary ostia obstruction. FIG. 1E shows incorrect placement, where the device is too below the annulus, resulting in perivalvular leaks. FIG. 1F shows yet another example of incorrect placement, causing both coronary artery ostia obstruction and perivalvular leaks.

An advantage of this invention is to securely place the prosthetic valve below the coronary ostia.

An advantage of this invention is to atraumatically attach the prosthetic valve to aortic leaflets and/or aortic wall. This, without excessive expanding radial forces against the aortic wall, or cause distortion of the aortic structure by having a very stiff and large prosthetic valve frame, in an attempt to leaks or migration.

An advantage of this invention is that the prosthetic valve is primarily secured to the native leaflets and gently to the aortic base/annulus, allowing the prosthetic valve to be less rigid, more compliant, and better retain the natural elasticity of aortic wall compliance/motion/movement.

An advantage of this invention is that the prosthetic valve frame has replaceable or removable leaflets, to address any calcification or other loss of functionality of the leaflets during procedure, post procedure-acute and/or chronic (>1 day, >30 days, and/or >100 years).

An advantage of this invention is that the arms securing to the leaflets are actuatable (independently or simultaneously), with or without disturbing the body of the prosthetic valve.

An advantage of this invention is that prosthetic valve leaflets can be placed at the level of native leaflets. An advantage of this invention is that the prosthetic valve leaflets are secured to the frame similar to how the native leaflets are secure to the aortic wall/annulus, for improved hemodynamics akin to healthy native leaflets.

FIG. 2A shows an exemplary embodiment of this invention, where the actuatable arms and the native tissue interfacing frame have atraumatic frictional elements, designed to engage with the native leaflets and/or the aortic wall.

FIG. 2B shows an exemplary embodiment of this invention that has a modular replaceable insert comprising of leaflets, within the frame that engages with the native leaflets and aortic wall. The insert can be reversibly attached using common techniques, such as, press-fit, twist fit, screw fit, chemical, electrical, and or magnetic fit.

FIG. 2C shows an exemplary embodiment of FIG. 2B, additionally comprising of an atraumatic annular grasping/engaging feature. At this location, and/or external to the prosthetic frame, a skirt comprising of sponge, gel, fabric, film, mesh, braid, balloon, and/or fibers may be used to augment prevention of perivalvular leaks.

FIG. 2D shows an exemplary schematic embodiment of this invention as in FIG. 2A, however, with prosthetic leaflets attached/extending below the prosthetic frame.

FIG. 2E shows an exemplary schematic embodiment of this invention as in FIG. 2B, however, with prosthetic leaflets insert attached/extending below the prosthetic frame.

FIG. 2F and FIG. 2G show an exemplary implantation of embodiments shown in FIG. 2A and FIG. 2C, respectively, in an aorta (schematic).

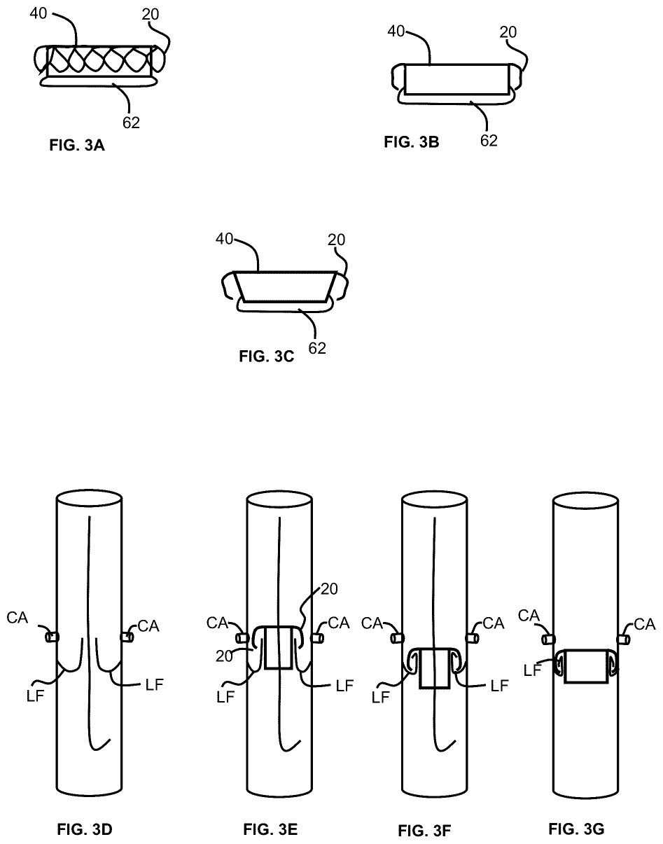

FIG. 3A to FIG. 3C show exemplary embodiments of this invention with actuatable arms and skirts.

FIG. 3D to FIG. 3G show an exemplary method of implanting the prosthetic valve. FIG. 3D shows schematic of an aortic valve. FIG. 3E shows the prosthetic valve body in a narrow constrained condition, while the arms are extended out, allowing for a leaflet insertion space. FIG. FIG. 3F shows the prosthetic valve over the native leaflets LF. Finally, the body of the prosthetic valve is expanded (self-expanding or balloon expanded), to securely and atraumatically engage with the native leaflets, as shown in FIG. 3G.

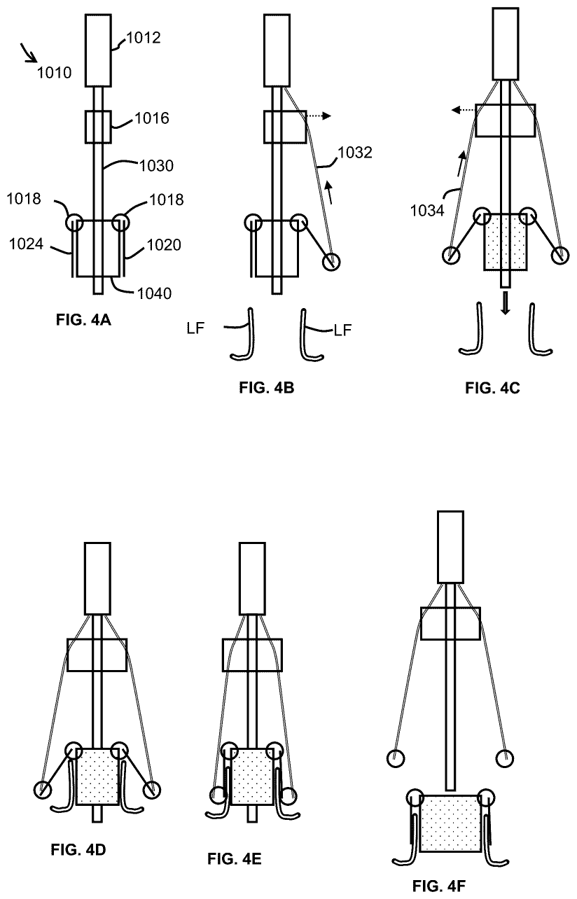

FIG. 4 shows an exemplary embodiment and method of this invention, wherein, the arms are actuated independently to securely and atraumatically capture the native aortic leaflets. FIG. 4A shows an exemplary schematic of an aortic prosthetic device 1040 with leaflet grasping arms 1020 and 1024. Note, only two leaflets LF and two arms are shown for simplicity. Typically for aortic, there will be 3 arms and 3 leaflets. The prosthetic device may have at least one arm to grasp at least one leaflet. The arms can be attached to the device 1040 via a feature 1018. The feature 1018 can be suture, a hinge, weld, glue, screw, and/or a rivet. The arm can be elastic, superelastic, shape memory, rigid and/or flexible material that can configured to be biased towards the prosthetic device. It can be made of nitinol, metal, plastic, ceramic or their combination. There is lever arm 1016, used to provide the required mechanical advantage to actuate the arms 1020, 1024 to create a separation to allow a leaflet to be inserted, as shown in FIGS. 4B and 4C. The lever arm can be swinging, bending, expanding, hinged arms, motor, and or any means known to POSA, that can actuate the arm, preferably with some mechanical advantage to actuate or apply biasing force/motion to open the arms, while allowing the lever arm feature to be retracted inside the delivery catheter profile. Note: there can be additional mechanisms to actuate the lever arm itself, so as to transform/translate the lever arm from low profile (position 1: to be contained within the catheter profile) to position 2 (expanded position to better exert the forces needed to actuate the arms 1020, 1024. The lever arms 1046 themselves can be configured to be individually or simultaneously actuatable. Each of the arms can be configured to be actuated individually or simultaneously. The device 1040 is then advanced over the leaflets as shown in FIG. 4D and the leaflets are captured by dropping the arms over the leaflets, as in FIG. 4E. The arms or the device can have barbs or other frictional elements to securely and atraumatically grasp the leaflets. The device can be repositioned, redeployed, or removed, by re-actuating the arms 1020, 1024, repeatedly, if necessary. The device can then be separated from the delivery system. In some alternate embodiments, the device can be reversibly separated from the delivery catheter by having retrieval features on the device 1040.

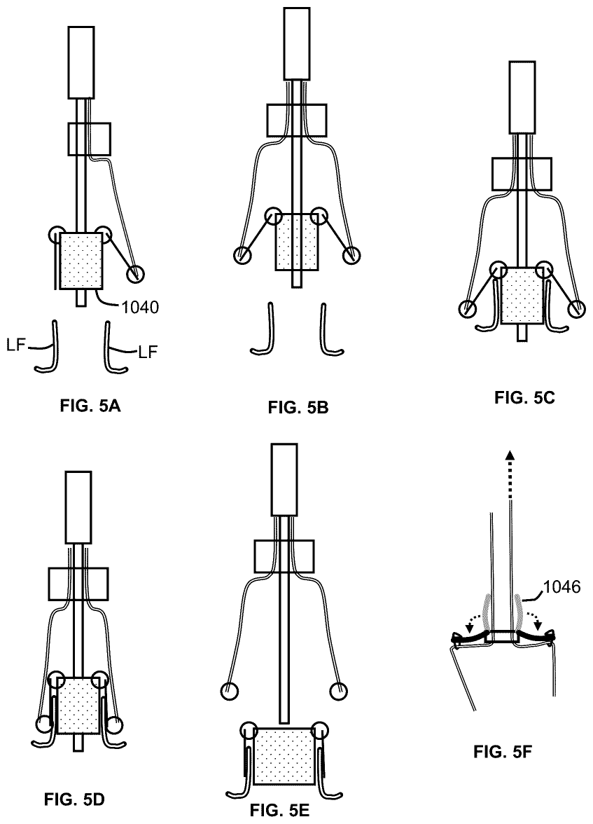

FIG. 5 shows an alternate embodiment similar to FIG. 4, with actuatable lever arms 1046, with a variation where is actuating suture is below (distal) to the lever arm position. FIG. 5F shows an example of lever arm 1046 configuration, where, the lever drops/swivels to provide the necessary lever arm to exert biasing forces to the arms 1020, 1024.

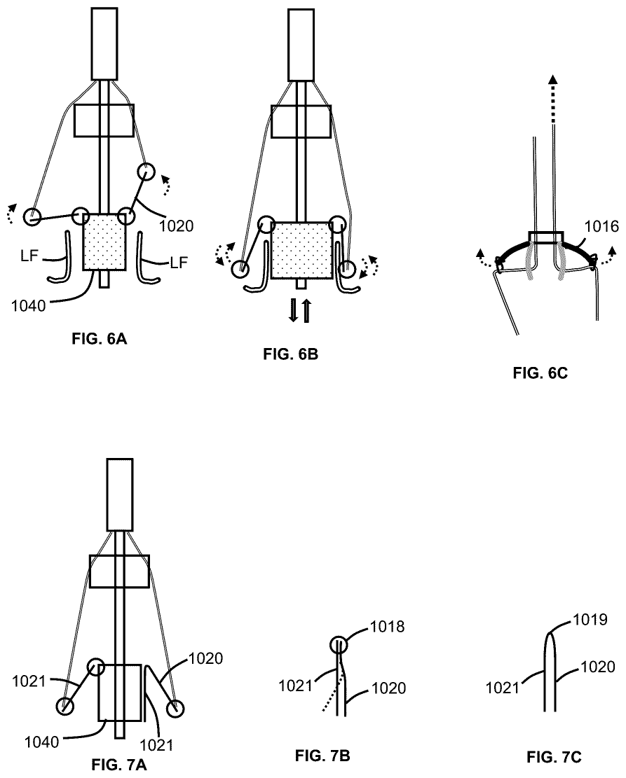

FIG. 6 shows an alternate embodiment of the invention shown in FIG. 4, wherein, the native aortic leaflets can be regrasped, repositioned, redeployed (FIG. 51E) and evaluated/assessed multiple times in the fully expanded state of the prosthetic valve body. Furthermore, the arms can have a large range of motion for superior placement, bailout, removal, and/or retrieval of the device, as shown in FIG. 6A and FIG. 6B. FIG. 6A shows arm manipulations in fully expanded state of the aortic prosthetic device 1040 with leaflet grasping arms 1020 and 1024. Note, only two leaflets LF and two arms are shown for simplicity. Typically for aortic, there will be 3 arms and 3 leaflets. However, in some embodiments, more than 1, 2, 3, 4, . . . , 99, and/or 100 arms may be used. In some embodiments, the arms can be actuated up to an inverted position, as shown in FIG. 6A. FIG. 6C is an exemplary embodiment of the lever arm, that needs to be raised/actuated/swived (outside of the catheter profile), to provide the necessary mechanical advantage. Any means can be used to raise/rotate it, such as a suture, screw mechanism, hydraulic, electrical, chemical, pneumatic etc.

The arms 1020, 1021, 1024 can be multiple components as shown in FIG. 7A. FIG. 7B shows arms 1020, 1021 that are two components fastened via 1018 and configured to be biased towards each other. For example, in one configuration, the arm 1020 can be made of shape-set, superelastic material such as nitinol and the dotted line of the arm 1020 shows the unbiased shape-set position. The arms can be attached at their base or anywhere between the arm and the device. FIG. 7C shows an example, where both 1020 and 1021 are made of continuous material/structure.

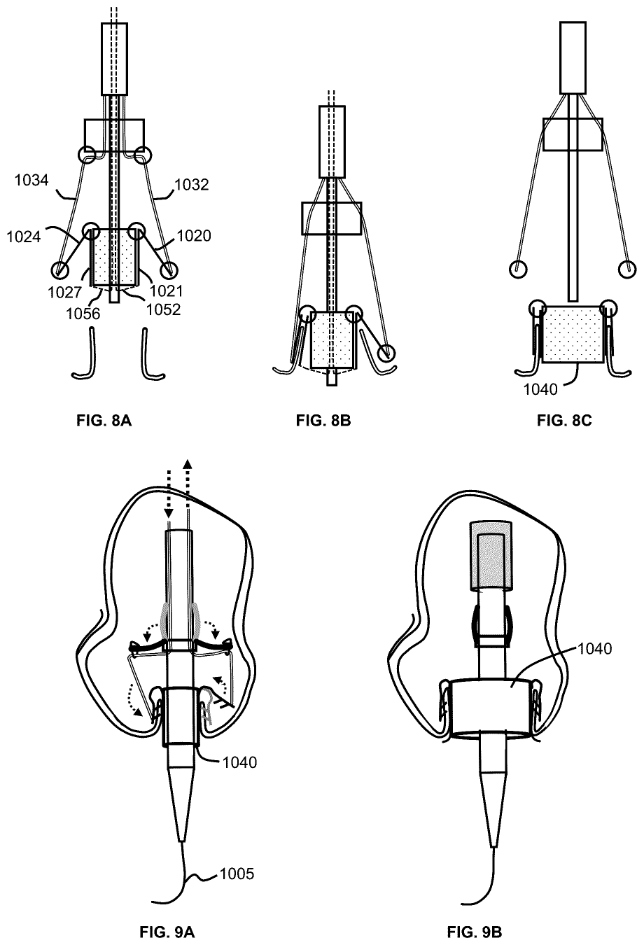

FIG. 8 shows an alternate embodiment of device 1040, wherein, it is configured to have a pair of arms (1021, 1020 and 1024, 1027) to grasp form both outer 1020, 1024 and inner 1021, 1027 side of the leaflet. These arms can be mechanically (via hinges, gears, etc) biased or self-biased elastically, example using nitinol. See co-owned prior arts for further description. In this embodiment, the arms are made of nitinol. Suture 1032 is used to actuate outer arm 1020 and suture 104 is used to actuate inner arm 1021, while suture 1034 is used to actuate outer arm 1024 and suture 1056 is used to actuate inner arm 1027.

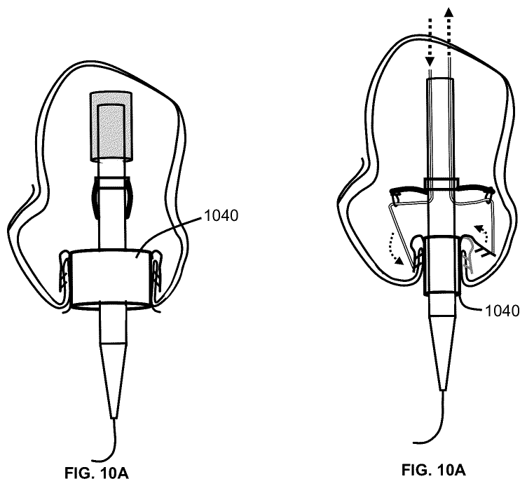

FIGS. 9A and 9B show exemplary embodiment of lever arm shown in FIG. 5F

FIGS. 10A and 10B show exemplary embodiment of lever arm shown in FIG. 6C

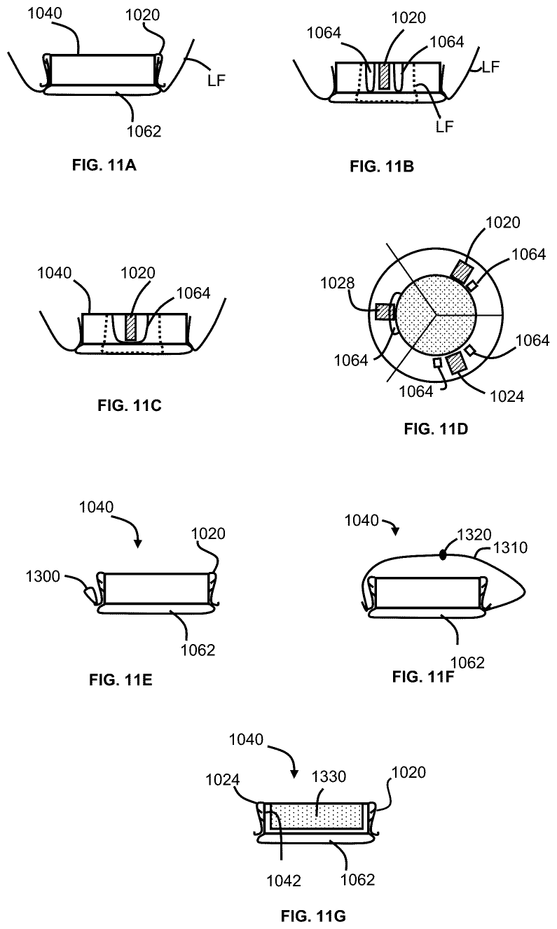

FIG. 11A shows a prosthetic device 1040 with actuatable arms with a skirt 1062 to prevent perivalvular leaks. The skirt can be hollow, balloon, stent with a cover, or any structure with a cover, an expandable and compressible structure, perforated structure, fabric, braid, film, coating, cushion, sponge, sponge like structure, or any structure that can prevent blood leaks. FIG. 11B, 11C, 11D show the example of prosthetic valve with various embodiments of position markers 1064, to aid in grasping by arms 1020, 1024, 1028 and implantation, similar to Jenavalve U.S. Pat. No. 10,575,947B2.

FIGS. 11E and 11F show exemplary inventions, wherein, the valve implant has one or more valve retrievable features 1300, 1310, 1320 to aid removal of the valve 1040 at a later date, post implantation (post complete detachment from the delivery system). The retrievable features 1300, 1310, 1320 can be suture loop, a hook, magnet, wires, threads, or any commonly known features that aid retrieval of device post implantation (see co-owned patents for additional details). These retrievable features can optionally have features that aid in detection, for example radio-opaque, echogenic, magnetic, florescent, etc. The features can be of polymer, metal, ceramic, nitinol, etc. The retrievable features can also be actuatable, deployable, remotely triggerable, in order to have low profile when not needed and easily graspable or detectable when needed (for easy retrieval). In some embodiments, the retrievable features are used stabilize the frame.

FIG. 11G shows an exemplary invention, wherein, the prosthetic valve 1040 comprises of valve housing 1042 and replaceable valve 1045. The valve housing can typically comprise of primary fixation features that are more of less fixed to the tissue post implantation, for example: the frame 1042, arms 1020, 1022, 1024, 1026, skirt 1062 etc. The replaceable valve comprises 1045 of the prosthetic leaflets with replaceable frame 1330. Additionally, the replaceable frame may comprise of retrievable features. In some embodiments, the retrievable features are on both valve housing 1042 and replaceable valve 1046. For example, the studs, hooks, or slots in the valve housing 1042 may be used to stabilize it, while the retrievable feature on the replaceable valve 1045 can be used to detach, remove, and replace it. In one alternate embodiment, the replaceable valve 1045 has tearable valve leaflets and secondary frame, to easily remove failed prosthetic valve leaflets. One advantage of this invention is that it allows for easy repair, in event of any failure of the valve in a future date, post implantation (or during implantation). This, to mitigate the need for cutting of failed prosthetic valve or removal of the entire failed prosthetic valve, which will require cutting of surround tissue, including the native leaflets.

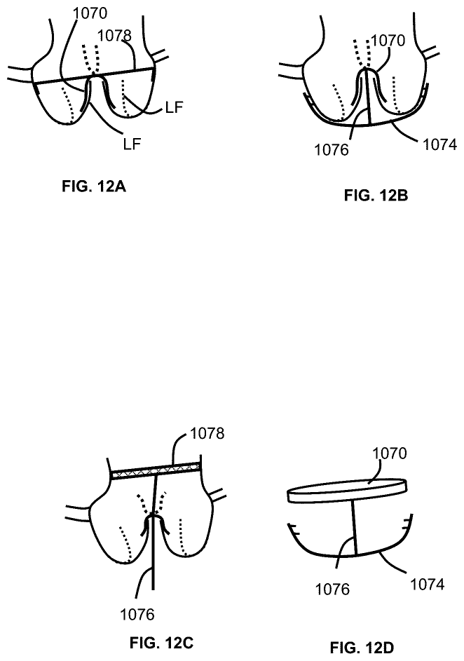

FIG. 12A shows a prosthetic device 1078 with a flexible tarp like feature 1070, which can cover the gap between the leaflets to mitigate regurgitation. The tarp can be flexible, rigid, stretchable, non-stretchable, hollow, balloon, stent with a cover, or any structure with a cover, an expandable and compressible structure, perforated structure, fabric, braid, film, mesh, fibrous, coated, cushion, sponge, sponge like structure, or any structure that can mitigate valve regurgitation when placed over the gap between the leaflets. The tarp can be made of metal, plastic, biological origin, non-biological origin, ceramic, superelastic, shape memory, composite, fabric, braid, machined, 3d printed, or any combination known to POSA. The tarp can comprise of at least one structure, 2 structures or 3 or more structures. It can have single leaflet covering, double leaflet covering, or 3 or more leaflet covering. It can be of any shape, circular, pizza, triangular, trapezoid, square, oval, and so on. It can be corrugated, folded, smooth, fan like bladed structure, Japanese foldable fan like structure, with slits or without any slits. The tarp can have features, stiffening members, wires, sheets, along the rim and sides to configure and optimize the shape during diastole (to minimize regurgitation) and during systole (to maximize blood flow).

FIG. 12A shows a stent structure 1078 that engages with the wall/Orifice of the valve just below the coronary ostia or about the level of the leaflets and is configured to support the tarp 1070. In one exemplary embodiment, the tarp 1070 folds down to cover over the gap in between the leaflets during diastole to mitigate the regurgitation. In systole, the native leaflets open (dotted lines) and the tarp folds upward and away from the leaflets to allow blood flow. The folding of the tarp can be passive (moving along with the blood flow) or active (controlled remotely or using a logic, fluidics, or programed). In passive mode, the extent of folding or shape of the tarp can controlled by configuring the structure of tarp by adjusting flexibility/bending modulus at each zone/segment/slice/section of the tarp 1070.

FIG. 12B shows a variation of the exemplary embodiment shown in FIG. 12A, wherein, the tarp 1070 is supported via a low-profile connecting feature 1076. The feature 1076 can be a suture, wire, stent structure, tube, etc. Furthermore, the feature 1076 can be stiff, elastic, superelastic, flexible, bendable, telescoping, adjustable, smart controlled, etc. Furthermore, the low profile feature 1076 is supported with a feature 1074 that is configured to attach to the base of the aorta as shown. The feature 1074 can have barbs, clasps, crocodile teeth, or any grasping feature that is mechanically actuated (non-superelastic or steel based) or self-biased (shape memory, superelastic, nitinol based).

FIG. 12C shows a variation of the exemplary embodiment shown in FIG. 12A, wherein, the tarp 1070 is supported via a low-profile feature 1076, which in turn is supported with a stent structure 1078 above the coronary ostia or much above the tip of the leaflets.

FIG. 12D shows the exemplary device as shown in FIG. 12D. Although, the tarp is shown as a single disc shape, it can be of any geometry, optimized for patient specific disease (regurgitation).

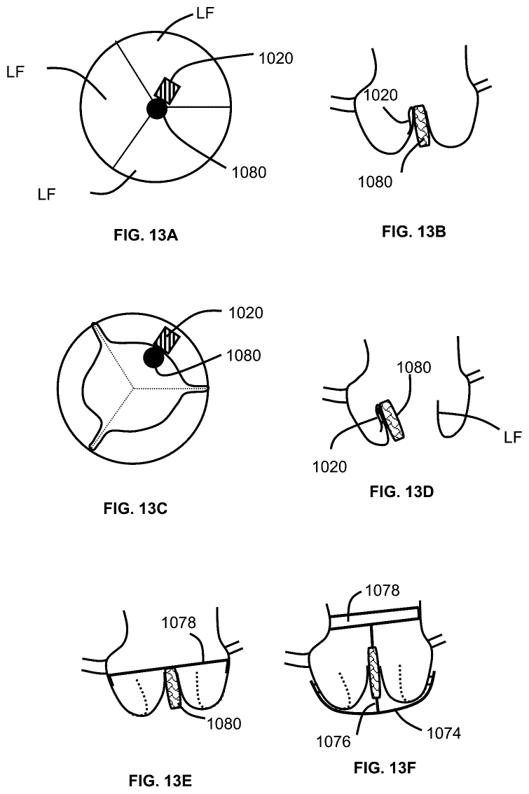

FIGS. 13A and 13B show an exemplary embodiment of a spacer 1080 with an arm 1020 during diastole. The spacer 1080 is configured to fill the gap to mitigate regurgitation. Although the shape of the spacer is shown to be cylindrical (with circular cross-section), the shape and cross-section can be any geometric shape, including a star, triangular, flat, crescent, ellipsoidal, etc. Further, the spacer can be expandable, compressible, stretchable, inflatable, porous, solid, wire form, sponge, to name a few examples. Lastly, the spacer can be smart, and the shape may be changed remotely electronically or hydraulically, or mechanically or chemically but not limited to these examples. For example, in one exemplary embodiment, the spacer may be controlled to expand to mitigate regurgitation in diastole and shrink to maximize flow during systole.

FIGS. 13C and 13D show the exemplary embodiment of FIGS. 13A and 13B during systole respectively.

While the exemplary embodiments in FIG. 13A to 13D show a single spacer device attached to a single leaflet, there can be more than one spacer device attached to a leaflet. Furthermore, more than one leaflets may be attached to 1 or more spacer devices.

FIGS. 13E and 13F show the spacer 1080 attached to various stent 1078, 1074 configurations. The stent 1078 is at the level of the leaflet tip in FIG. 61E. The spacer 1080 is attached via feature 1076 to both stents 1078 (above the leaflet tip and stent 1074 below the leaflet.

In an alternate embodiment, the spacer 1080 may be replaced with a prosthetic valve 1040, configured to mitigate regurgitation.

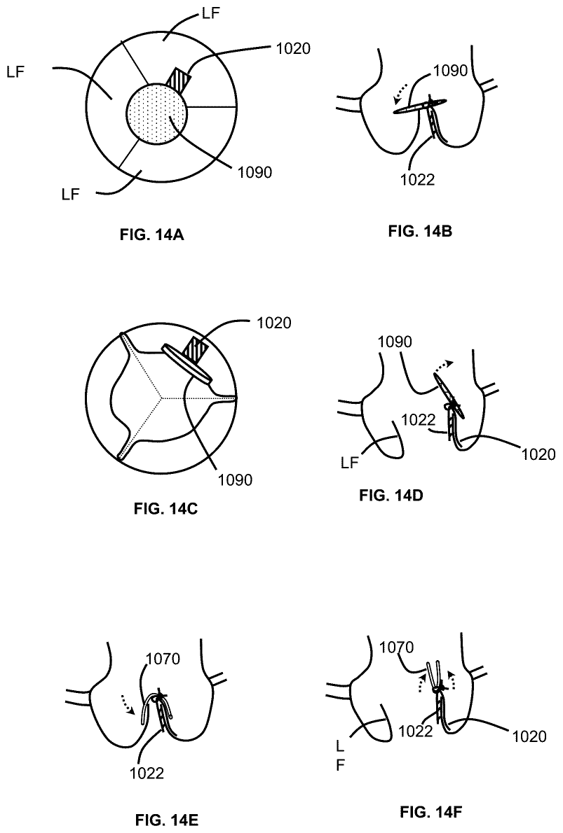

FIGS. 14A and 14B show an exemplary embodiment of a lid 1090 with an arm 1020 during diastole. The lid/tarp/disc 1090 is configured to fill the gap to mitigate regurgitation. Although the shape of the spacer is shown to be cylindrical (with circular cross-section), the shape and cross-section can be any geometric shape, including a star, triangular, flat, crescent, etc. Further, the spacer can be expandable, compressible, stretchable, synthetic, 3d printed, molded, extruded, fabricated, layered with films, reinforced composite structure, inflatable, porous, solid, wire form, sponge, to name a few examples. Lastly, the spacer can be smart, and the shape may be changed remotely electronically or hydraulically, or mechanically or chemically but not limited to these examples.

FIGS. 14C and 14D show the exemplary embodiment of FIGS. 14A and 14B during systole, respectively.

While the exemplary embodiments in FIG. 14A to FIG. 14D show a single lid device attached to a single leaflet, there can be more than one lid device attached to a leaflet. Furthermore, more than one leaflets may be attached to 1 or more spacer devices.

FIGS. 14E and 14F show an exemplary embodiment of the tarp/film 1080 attached to arms 1020, 1022, attached to a leaflet in diastole and systole, respectively.

As POSA may understand, the embodiments described in this invention for Aortic Valve, can be applied to other heart valves (for example, mitral, tricuspid, pulmonary valves). venous valves, or any other valves in the human body/robots/humanoids.

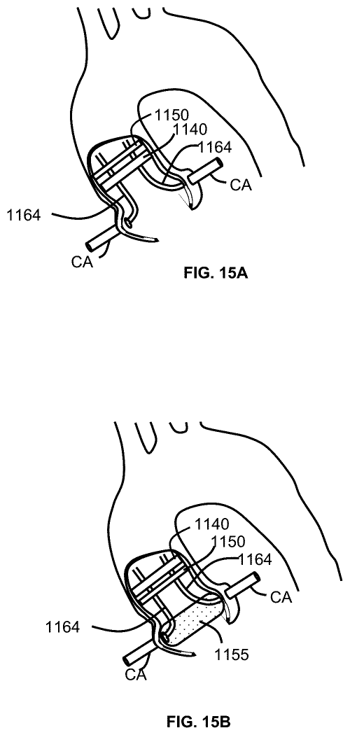

In aortic stenosis, the common cause is heavy calcification in the native leaflets. This leaves very little space for blood flow and/or restricts the motion of the leaflets. Likewise, prosthetic tissue valves fail in 7 to 15 years, typically due to leaflet calcification. Hence, it is beneficial to either remove the failed prosthetic valve completely or cut the prosthetic leaflets (entirely or partially, prior to replacing it with a new prosthetic valve. However, once the calcified leaflets are cut, the blood flow and pressure will be compromised until the new prosthetic valve is deployed/implanted. A temporary valve can be placed either downstream or upstream of the coronary sinus to mitigate this issue. If the temporary valve is placed upstream to the valve (or coronary sinus), then the blood flow to the coronary arteries are not compromised. If the temporary valve is placed downstream to the coronary sinus, then, there needs to be a way to supply blood flow to the coronary arteries. In either case, it is critical to ensure that the blood flow to the coronary arteries and downstream organs is devoid of any debris or thrombus or emboli that can form during resection of the diseased native valve or failed prosthetic valve. This can be achieved by placing a downstream filter immediately after the valve, for example at the ascending aorta and before the coronary sinus. Alternatively, the filter can be placed downstream of the coronary sinus, in which case, debris free blood to the coronaries can be provided using tubes 1164, as shown in FIG. 15A. As it is obvious to POSA, the seal of the blood flow to coronary may be achieved using commonly known means, including a snug fit tube, tapered tube, inflatable or spongy cuff, a stent, O-ring, etc. FIG. 15B shows an exemplary balloon cuff 1155.

Furthermore, as POSA can understand, the exemplary configuration of embodiment of FIG. 15A, the positions of temporary valve 1140 and filter 1150 can be interchangeable, that is, the filter 1150 may be placed immediately upstream to the temporary valve 1140, as shown in FIG. 15B. Alternatively, the functions of filter and temporary valve can be combined into a single embodiment. In an alternate embodiment of the temporary valve, its function can be changed from being a one-way valve (flow in single direction) to a two-way valve (flow in both directions). This can be achieved mechanically, electrically, electronically, chemically, magnetically, using software, fluidic trigger, etc. The same valve can be switched/transformed/changed to always remain in open position (allowing flow in both directions) or perform normally as a check valve (open in forward direction flow and closed in reverse direction flow. Alternatively, the temporary valve may have any configuration obvious to POSA that allows for selectable flow control.

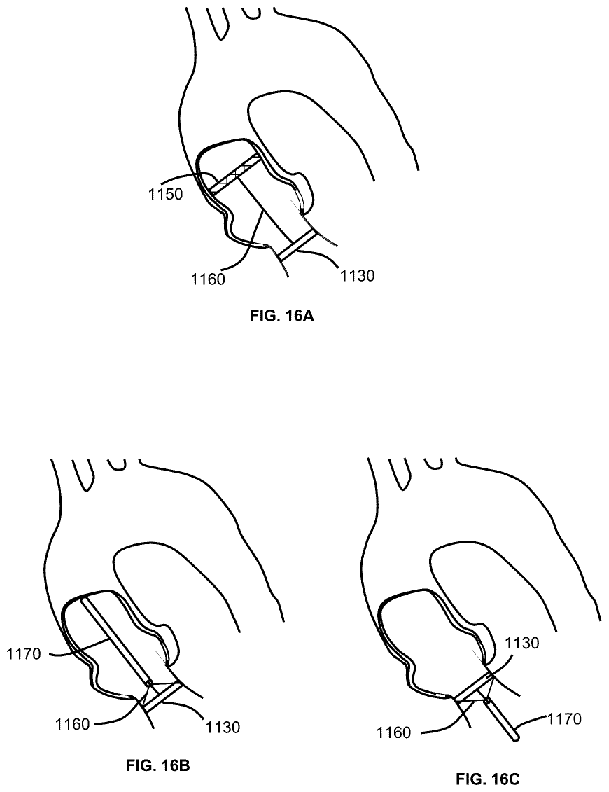

Temporary valves can be placed FIG. 16A to 16C show various configurations of temporary valves that are placed upstream of coronary sinus, either immediately below the native valve or in between the coronary artery ostia, CA, and native valve. This ensures that flow to coronary arteries is not compromised. Similar to FIGS. 15A and 15B, filters may be used to mitigate embolic debris during the procedure (note shown here for simplicity).

FIG. 16A shows a upstream temporary valve 1130 that is stabilized using a stent/filter 1150 and an interfacing member 1160. Note, the stent can be hollow, incorporate filter, ports, a secondary valve (in addition to 1130). The interfacing member 1160 can be stiff, flexible, elastic, plastic, superelastic, straight, curved, single wire, multiple wires, braid, coil, laser cut, metal, polymer etc. and can be detachably attached to both stent 1150 and temporary valve 1130, 1140. FIG. 16B shows an alternate embodiment, wherein, the temporary valve 1130 (or 1140) is stabilized/supported or held in place using a catheter shaft 1170, with or without comprising interfacing member 1160. The catheter is coming from retrograde direction. FIG. 16C shows an alternate embodiment, wherein, the catheter shaft 1170 is coming from the ventricles (antegrade). In an alternate embodiment, the temporary valve may have a combination of the embodiments shown in FIG. 12-14.

One advantage of this invention is that the temporary valve 1140, 1130 can be configured to continue to function as a permanent valve. One advantage of this invention is that any damage on removal of the diseased native valve or failed prosthetic valve can be mitigated by placing the prosthetic valve downstream or upstream to the native valve, where the tissue is relatively free of damage or has more suitable anatomy. As POSA may understand, the temporary filter and temporary valve position may be interchangeable or incorporation in the same structure, and is used only for short term, and may be removed at the end of the procedure or at a later date.

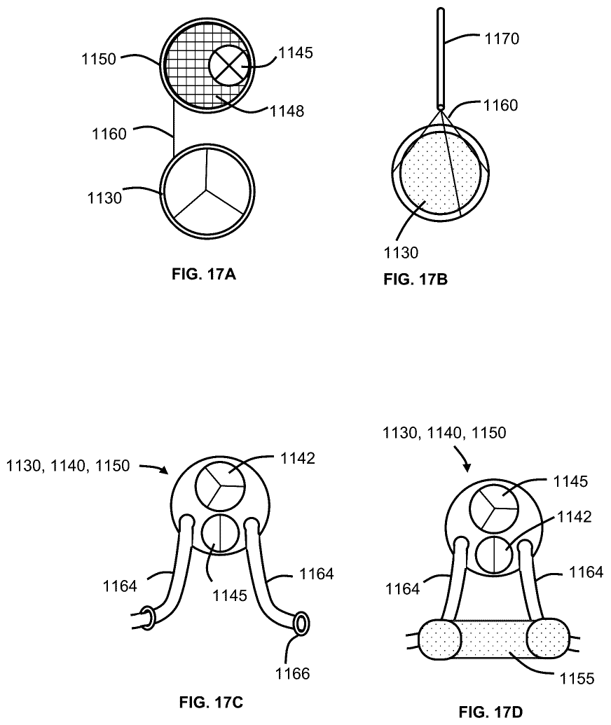

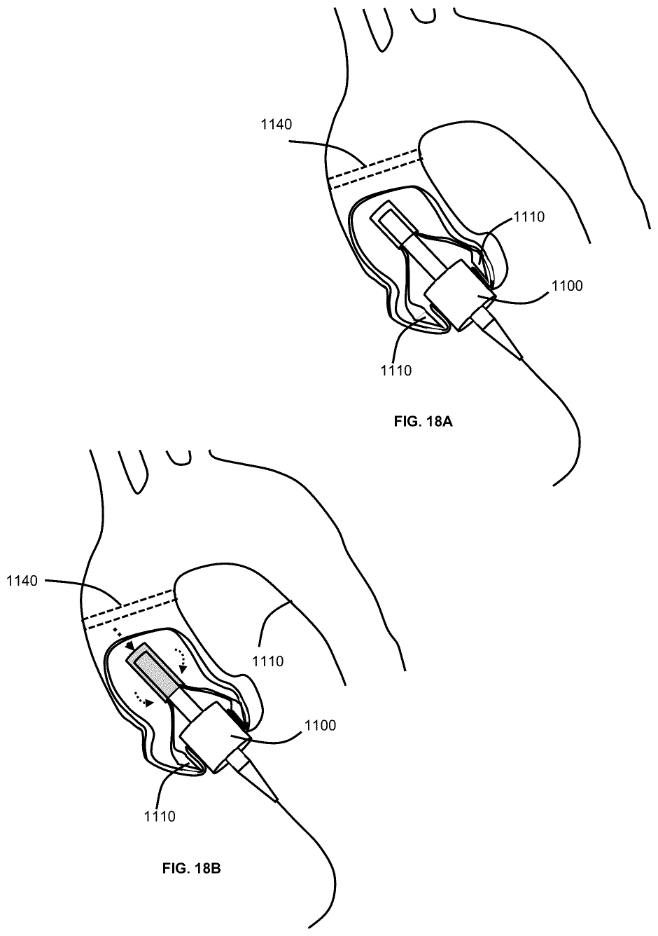

FIG. 17A shows exemplary embodiments of the filter 1150 and temporary valve 1130. The filter comprises of a mesh 1148 and a self sealing or sealable port 1145 to advance various catheters. The mesh can be any known filters, that are commonly used in medical device industry. The port 1145, can be any commonly known means in the catheter industry that allows passage of catheters, such as a self-sealing film, barrier, or valve. The temporary valve 1130 can be a tri-leaflet design, mono-leaflet, bi-leaflet, or more than 1 leaflet valve, a check valve, a duck-bill valve, a disc valve, a cone valve, a low-profile valve, etc. Alternatively, it too can have port similar to port 1145 to allow for passage of a catheter, while maintaining the pressure differential across the port/valve. Alternatively, the filter 1148, 1150 can be inside the catheter, when the blood is configured to flow through the catheter.

FIG. 17B shows an exemplary embodiment of the temporary valve 1130 with stabilizing catheter 1170 and interfacing member 1160.

FIG. 17C shows an exemplary configuration of the temporary valve 1130, 1140, 1150, comprising of coronary artery feeding tubes 1164 and a sealable or self-sealing catheter port 1145 and a one-way valve 1142. Seal between the tubes 1164 and coronary sinus can be achieved using any of the following exemplary means/methods: snug fit tubing, longer length tubing, conical tubing, O-ring based concept, expandable, inflatable, fillable, compressible cuff, balloon, suture, clip etc., known to the POSA.

FIG. 17D shows the seal at the coronary sinus is created using donut like balloons, wherein, the tubes are passing through the cross-section of the balloon. Also, the size and the form of the ports and valve configuration can be interchangeable.

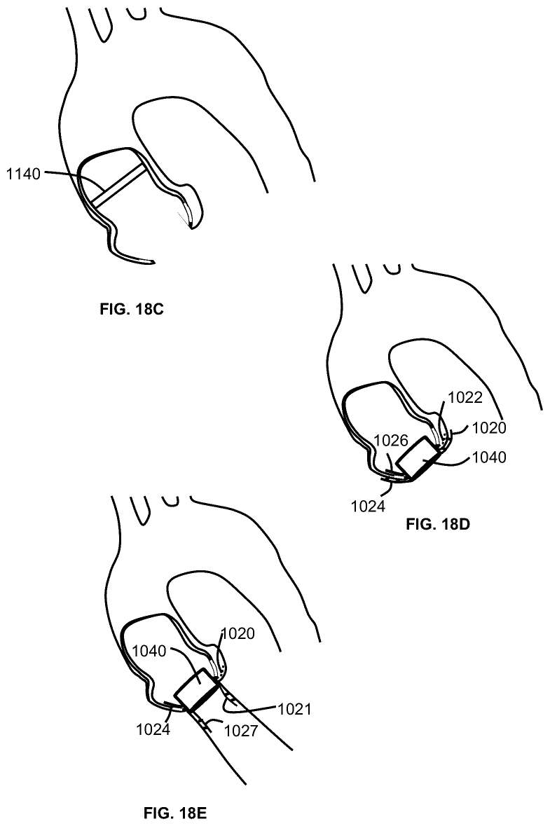

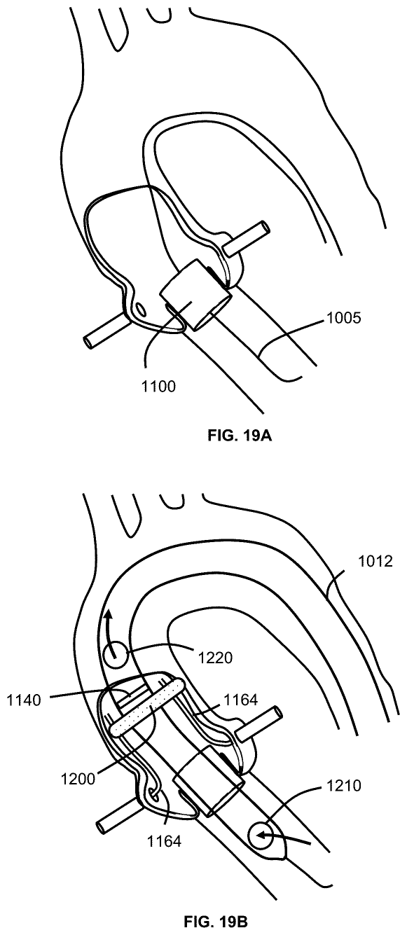

FIG. 18A shows a variation of the exemplary invention, wherein, a previously deployed prosthetic valve 1100 is removed and replaced with a new prosthetic valve 1040. FIG. 18A shows placement of a temporary valve 1140 (note filter 1150 and tubes 1164 are not shown for simplicity) and advancement and placement of a cutter device 1110 towards the existing prosthetic valve 1100. In FIG. 18B, the existing prosthetic valve is excised using any means commonly known to POSA, such a cutting, coring, punching, shaving, burring, grinding, RF, US, ablation, wire cutting, liquid jet, fluid jet, laser, chemical ablation, etc.

FIG. 18C shows the temporary valve in place and the existing prosthetic valve excised and removed. In this exemplary embodiment, a significant portion of the native valve leaflets are cut too. Alternatively, the leaflets may be cut partially, to allow for increased grasping via arms. FIG. 18D shows placement of a new prosthetic valve 1040, secured with arms 1020, 1021, 1022, 1024, 1026, 1027 that grasp the leaflets (or anatomy beyond the leaflets) from both sides. However, as evident from the invention, there can be various ways to attach, including using a clip, suture, barbs, cork screw, pins, adhesive, fusion achieved via chemical, electrical, mechanical, ultrasound, etc. FIG. 18E shows an alternate method of grasping and securing the valve. Any of the valves in this invention (temporary or permanent) may comprise of a one way valve, a port, a tube. The can be of low profile designs, including and not limited to the designs derived from mechanical valves, tissue valves, industrial valves. The valves can be self-expanding, expandable, compressible, bendable, twistable, shrinkable, etc to enhance compliance with catheter based delivery.

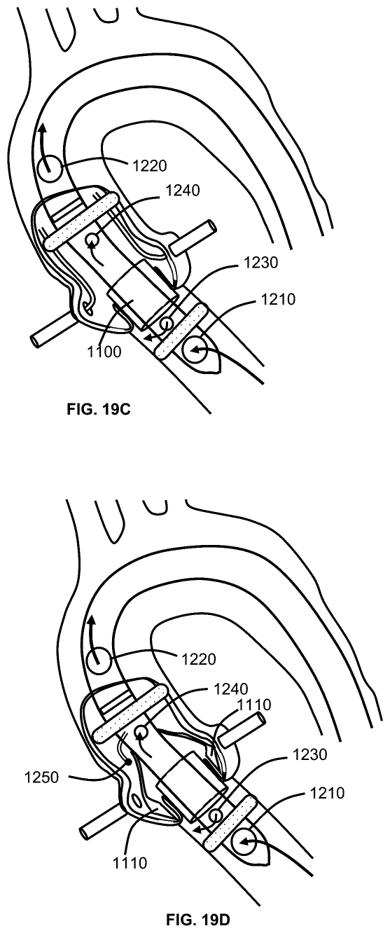

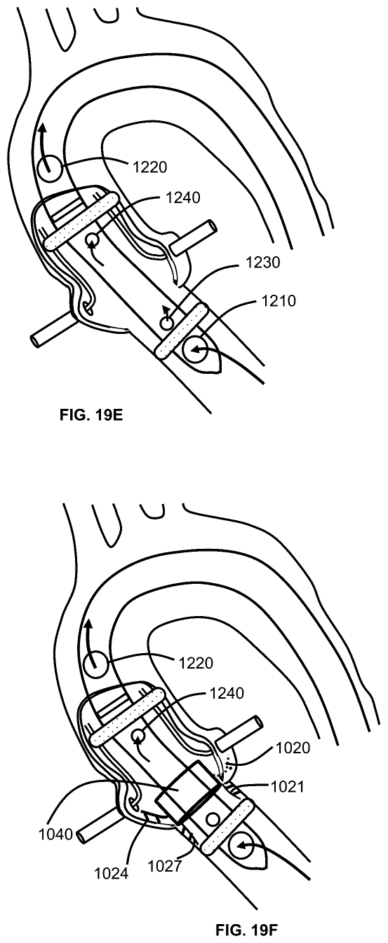

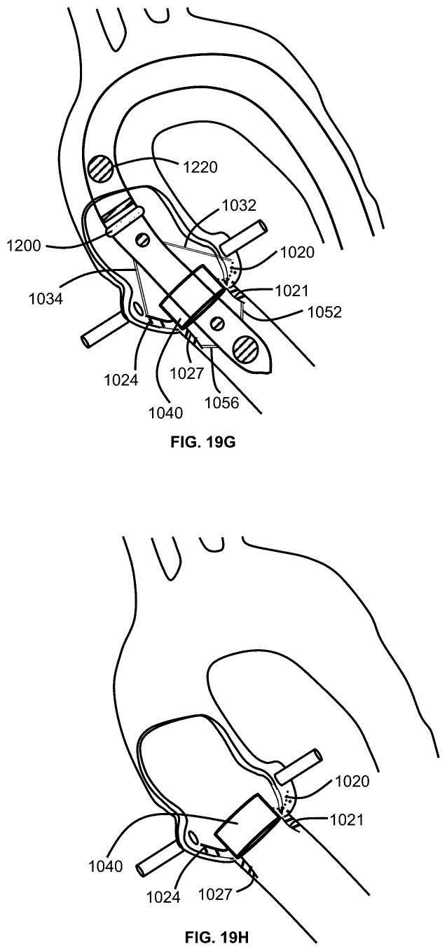

FIG. 19 shows a method of replacing failed aortic valve with new aortic valve with or without visualization. FIG. 19A shows a guidewire 1005 through the failed valve 1100.

FIG. 19B shows catheter 1012 inserted over the guidewire. Temporary valve 1140 and optionally filter 1150 can be placed inline, Cuffs 1200 are deployed. Note, the Cuff 1200 can be balloon, hemispheric, concave, cup shaped, bowl shaped, expandable umbrella like structure, disc, stent, or any feature used to restrict blood flow. Further, the cuff may have additional ports to allow multiple catheters, external to catheter 1012. Optional and exemplary conduits 1164 may be used to maintain blood supply to the coronary arteries. In addition, the Cuffs 1200 and conduits 1164 may be alternatively placed/deployed using a separate, independent, and dedicated catheter, prior to or after insertion of the catheter 1012 or along with it (working in tandem). Alternately, in place of the cuff 1200, a temporary valve 1140 and filter 1150 can be deployed outside of the catheter. Any of the temporary valves and filters described in this invention can be incorporated inside the catheter with ports or holes to allow for blood flow and cuff or balloons seal along the blood vessel. Alternatively, a separate removable catheter (inside or outside of catheter 1012) with temporary valve, cuffs, may be used in parallel to direct and filter blood flow and provide a safe working zone, during removal of the failed valve 1100.

FIG. 19C shows an invention wherein, an additional cuff 1200 is placed and ports 1230, 1240 are used to flush the working zone around the failed valve (native or prosthetic) with clear, translucent, or transparent liquid such as heparinized saline, to aid visualization using cameras 1250 (obvious to POSA). The safe visualization method is not limited to optical, any other visualization technique commonly known in the medical device or engineering industry maybe used. POSA is aware that saline visualization if preferred, however, is an optional step that is redundant with normal imaging techniques such as echography, fluoroscopy, IVUS, ICE, infrared, OCT, etc. Optionally, in an alternate embodiment, the port hole 1210 1220, can comprise of an LVAD motor/pump to off-load the pressure across the aortic valve.

FIG. 19D shows an exemplary cutting tool used to resect the failed valve 1100. As POSA may appreciate, any known technique can be used to capture and remove the failed valve using transcatheter technique. The failed valve 1100 may be removed using a multiple catheters if needed from both antegrade and or retrograde path. In additionally, the failed device may be removed as a whole or in fragments. In one exemplary embodiment of this invention, only the leaflets of the prosthetic valve 1100 are cut and removed. In which case, the new prosthetic valve 1040 will be deployed inside the frame of the failed prosthetic valve 1100.

FIG. 19E shows the stage post removal of the failed device 1100. Note: One advantage of this invention is that once the temporary valve is in place, the patient can be recovered (pre or post removal of the failed valve 1100) and this procedure can be continued at a later time or day.

FIG. 19F shows placement of the new prosthetic valve 1040, using actuatable arms to enhanced fixation of the valve 1040. More than one arm (1020, 1021, 1024, 1027, or 1026) may be used. Additionally, the arms can be used in pairs (for example, 1020 and 1021) and more than one pair of arms (for example, 1024 and 1027, 1020 and 1021) may be used. However, as POSA will appreciate, this method can be used to deliver any traditional valve (without actively actuatable arms).

FIG. 19G shows an exemplary embodiment of the invention, wherein, the flow modifying cuffs 1200 are removed, disabled, or de-actuated to allow free flow of blood. This, to allow for realtime or traditional assessment of the new prosthetic valve. In an alternated method/embodiment, the catheter can be retracted from the prosthetic valve leaflets during assessment. In an alternate embodiment, larger segment of the catheter can be retracted, and replaced with a significantly smaller size catheter can be used to during assessment and subsequent repositioning or manipulation of the arms 1020, 1022, 1024, 1026, of the prosthetic valve 1040, to improve quality of assessment. A POSA is aware, debris filter can always be used during this entire procedure. Furthermore, the debris filter may be part of this system or an external commercial system or both.

In this step, the new prosthetic valve 1040 may be optionally repositioned, redeployed, or completely removed from the catheter (and replaced with a different (size or shape) valve if needed. The valve 1040 can be repositioned, redeployed, completely removed and replaced with a different valve, multiple times. Note: The flow modifying cuffs 1200, temporary valve 1140, filter 1150, ports 1210, 1220, 1230, 1240 can be deactivated/reactivated, disabled/enabled or opened/closed, inflated/deflated, turned on/turned off, deactuated/actuated multiple times if needed, to allow for assessment, manipulation, or removal/replacement of any of the valves 1040, 1100, during the procedure.

FIG. 19H shows the new prosthetic valve 1040, after removal of the delivery catheter 1012. Any other procedure supporting features, such as filters, temporary valves, sensors, or any embodiments described in this invention or known to posa, used to monitor, help in speedy recovery, fine tune the implant/valve or assess the healing maybe removed at a later time or date.

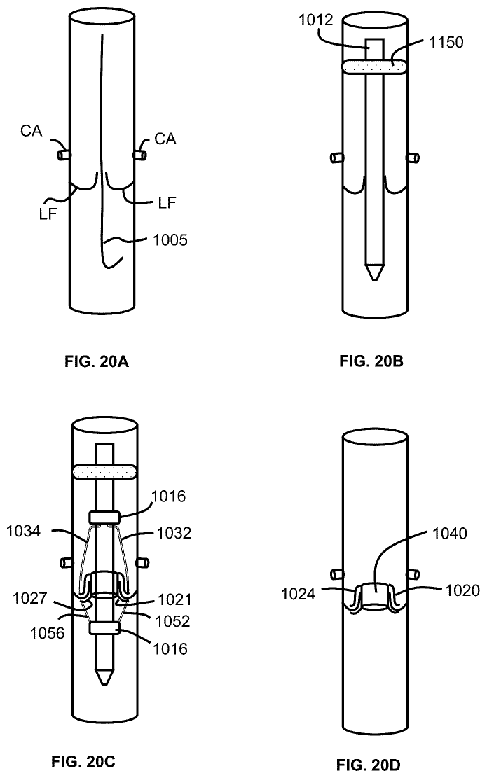

FIG. 20 shows a method of aortic valve deployment without cutting native leaflets.

FIG. 20A shows guidewire placed through the exemplary aortic valve schematic. While this exemplary method is shown in traditional retrograde approach to the aortic valve (from femoral artery access), this procedure can be alternatively performed from the antegrade approach (for example, from femoral vein access, via right atrium (trans-septal), left atrium, left ventricle to the aortic valve).

FIG. 20B shows insertion of the catheter 1012 over the guidewire 1005 (not shown for simplicity) and deployment of the filter 1150.

FIG. 20C shows placement of the prosthetic valve 1040. One advantage of this invention is that actuatable arms can be used as position markers and or for attaching to the leaflets. Additionally, dedicated position markers 1062 can be used along with the actuatable arms 1020, 1022, 1024, 1026. One advantage of this invention is that the arms can be independently or simultaneously actuated. One advantage of this invention is that the delivery catheter comprises of a deployable filter 1142. One advantage of this invention is that the filter can remain in place for extended period of time (preferably between 1 to 30 day or 1 to 180 days) post implantation and removed at a later time or date, to reduce or eliminate the need for blood thinners. One advantage of this invention is that traditional valve (without actuatable arms) can also be used. One advantage of this invention is that the valve can be assessed or repositioned both in compressed and fully expanded state. One advantage of this invention is that the prosthetic valve has retrievable features such as sutures, wire loops, hooks, (see co-owned patent for additional description), for ease of retrieval.

FIG. 20D shows the implanted prosthetic valve 1040. One advantage of this invention is the prosthetic valve 1040 can comprise of valve housing 1042 and replaceable valve 1045.

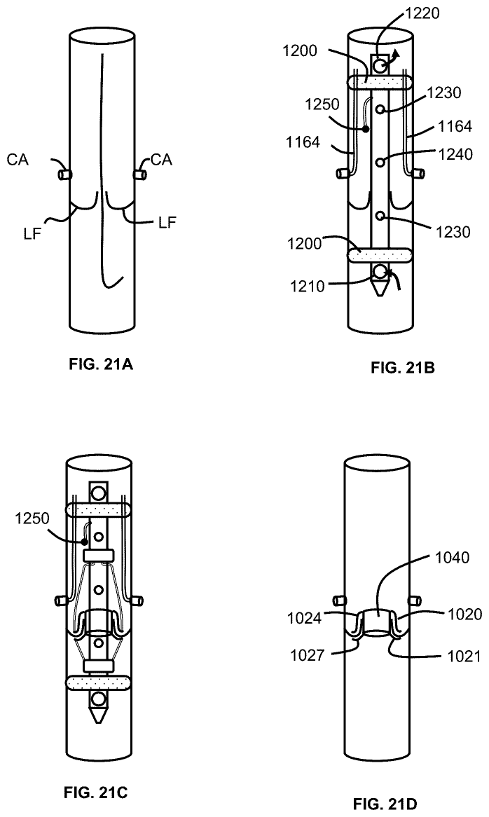

FIG. 21 shows a method of traditional deployment without cutting native leaflets and with visualization and blood flow management to the coronary arteries.

FIG. 21A is same as FIG. 20A.

FIG. 21B comprises of FIG. 20B and additionally comprises of ports 1210, 1220 with a temporary one-way valve 1140 embedded inside the catheter, cuffs 1200, and ports 1230, 1240, used to flush clear liquid such as heparinized saline. A camera 1250 can then be used to optically visualize the valve structures. Optionally, in an alternate embodiment, the port hole 1210 1220, can comprise of an LVAD motor/pump to off-load the pressure across the aortic valve.

FIG. 21C is similar to FIG. 20C with the addition of the ability to visually see the internal structures between the two cuffs 1200.

FIG. 21D shows the end result, similar to FIG. 20D.

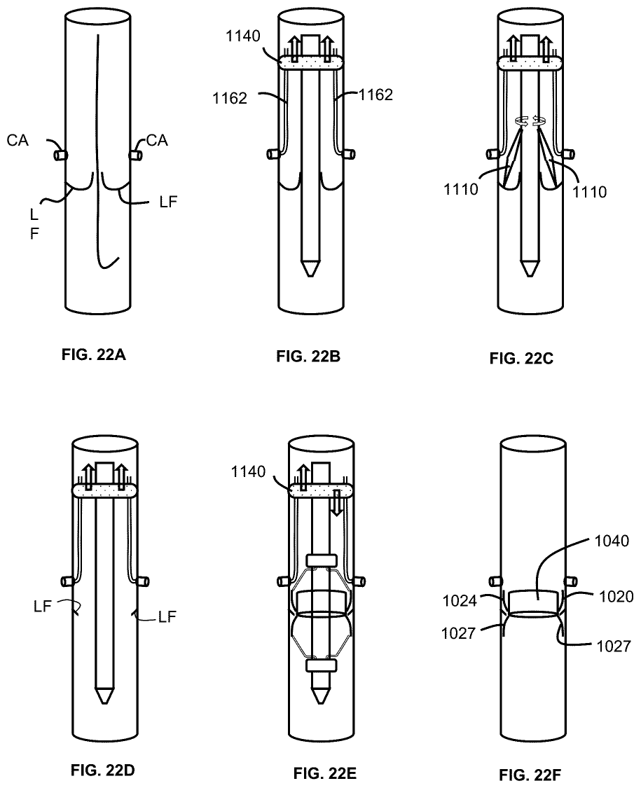

FIG. 22 shows an exemplary method of aortic valve deployment with cutting of native leaflets.

FIG. 22A is same as FIG. 20A.

FIG. 22B comprises of FIG. 20B and additionally comprises of temporary valve 1140, tubes 1162 to supply blood to coronary arteries. A filter 1150 may be optionally used.

FIG. 22C is similar to FIG. 20B, and additionally, the native leaflets are cut using blades 1110 for example and removed. Alternatively, laser, rf, ultrasound, chemical, or any combination of energy sources known to POSA may be used.

FIG. 22D shows the valve with cut native leaflets. The leaflets can be completely or partially cut.

FIG. 22E shows placement of the prosthetic valve 1040, by manipulating arms 1020, 1021, 1024, 1027. Similar to FIG. 20C, suture 1032 is used to actuate arm 1020 and suture 1034 is used to actuate arm 1024, suture 1052 is used to actuate arm 1021, and suture 1056 is used to actuate arm 1027. Once the physician is ready to test the efficacy of the valve, he can reversibly switch the temporary valve 1140 from being a one-way valve to a two-way valve, freely allowing the blood to flow in both directions.

FIG. 22F shows fully implanted prosthetic valve 1040, with an alternate arms configuration. One advantage of this invention is that more than 2 arms can be placed anywhere along the height of the valve.

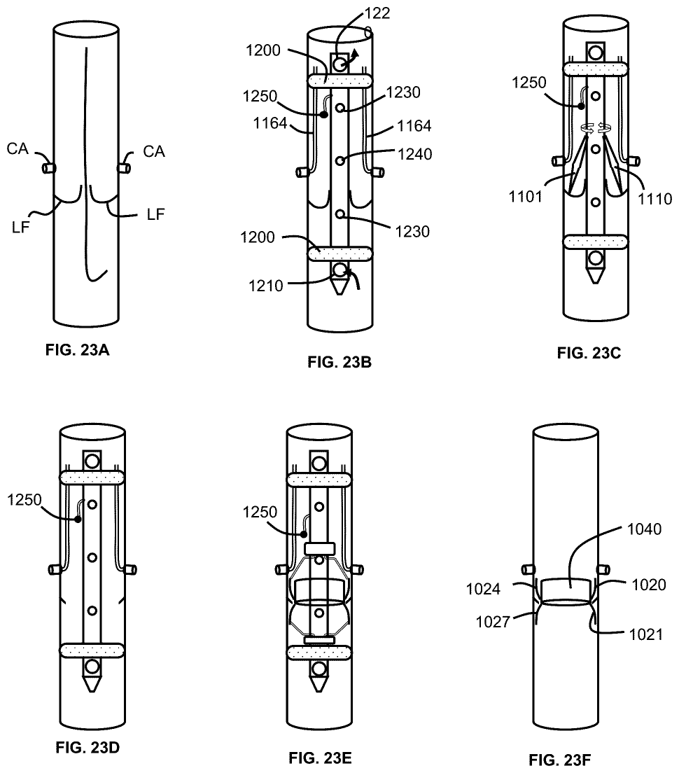

FIG. 23 shows an alternate method of prosthetic aortic valve deployment with cutting of native leaflets with saline flush for visualization.

FIG. 23A is same as FIG. 21A or FIG. 20A.

FIG. 23B is similar to FIG. 21B and comprises of ports 1210, 1220 with a temporary one-way valve through the catheter, cuffs 1200, and ports 1230, 1240, used to flush clear liquid such as heparinized saline. A camera can then be used to optically visualize the valve structures and or other structures between the two cuffs 1200. Optionally, in an alternate embodiment, the port hole 1210 1220, can comprise of an LVAD motor/pump to off-load the pressure across the aortic valve.

FIG. 23C shows the native leaflets are cut using blades 1110 for example and removed. This procedure can be performed under optical visualization.

FIG. 23D shows the valve with cut native leaflets. The native leaflets can be completely or partially cut and removed.

FIG. 23E shows placement of the prosthetic valve 1040, by manipulating arms 1020, 1021, 1024, 1027. Similar to FIG. 20C, suture 1032 is used to actuate arm 1020 and suture 1034 is used to actuate arm 1024, suture 1052 is used to actuate arm 1021, and suture 1056 is used to actuate arm 1027. Once the physician is ready to test the efficacy of the valve, he can reversibly switch the temporary valve 1140 from being a one-way valve to a two-way valve, freely allowing the blood to flow in both directions.

FIG. 23F shows fully implanted prosthetic valve 1040, with an alternate arms configuration. One advantage of this invention is that more than 2 arms can be placed anywhere along the height of the valve. Note: In case there is a failed prosthetic valve 1100, this same procedure can be followed to cut and remove the failed valve 1100 or alternatively, cut and remove only the prosthetic leaflets of the failed valve 1100.

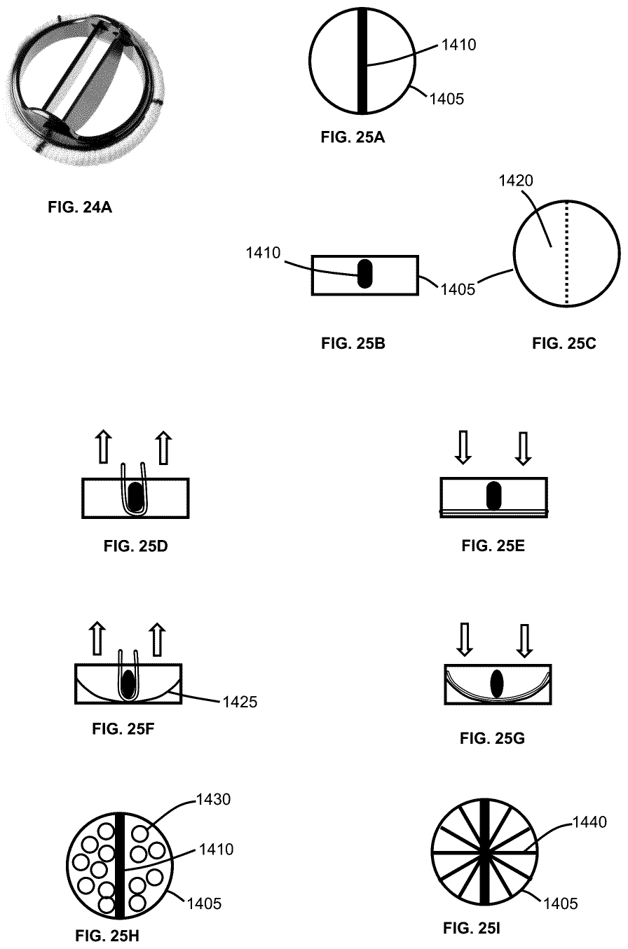

FIG. 25 shows a commercial mechanical valve. One of the issues with it is exposed metal. Despite being covered by carbon coating to improve hemocompatibility, the valve is still thrombogenic and required blood thinners for life. Furthermore, it makes annoying clicking noise and needs to be surgically implanted.

One of the exemplary embodiment of this invention is to cover the exposed metal with endothelium promoting coatings or coverings. For example: film, fabric, braid, mesh, woven, fibrous paper etc made of commonly known tissue growth promoting materials such as eptfe, pet or polyester in the form of, etc.

One of the exemplary embodiment of this invention is to replace the metal leaflets with polymeric or composite materials. The polymeric leaflet can be rigid, flexible, tarp like, silicone, rubber like, reinforced plastic, 3D printed, fabric, fiber/wire/nitinol reinforced fabric, and/or etc. These leaflets can optionally be coated or covered. Examples of composite materials can be fiber reinforced, nitinol reinforced base material such as fabric, metal, braid, ceramic, and/or plastic.

One of the exemplary embodiment of this invention is to decrease the travel of the leaflets to fully close, thereby, reduce the hammer effect and stress on the leaflets. For example, the hit of implant can be configured such that the leaflet close within 45 degrees of travel, preferably about 20 degrees.

One of the exemplary embodiment of this invention is to dampen the impact of leaflet closure forces or velocity, by adding silicone, rubber, spring, mesh, braid, fabric, or plastic dampeners. Creation of flutes, reliefs, friction, channels, dents, blisters, multiple holes or flow paths, and other dampening methods known to POSA.

An exemplary embodiment of this invention is replacing the metallic leaflets with flexible tarp like leaflet concept is shown in FIG. 25. FIG. 25A shows the top view of the frame with a stationary and rigid horizontal member. FIG. 25B shows the side view of the frame. FIG. 25C shows the flexible, tarp like, leaflet like material in the shape of disc. FIG. 25D shows the flexible disc like leaflet bend and allow flow of blood. FIG. 25E shows the disc below the horizontal member, blocking the blood flow. Thus, acting as a one-way valve. POSA will appreciate that the only moving component of this device is the flexible bending of the tarp 1420. Thus, eliminating the risks of friction, wear. Furthermore, the frame can be made rigid (for surgical implantation). The tarp 1420 can be removable attached or sutured or bonded to the horizontal member.

In an alternate embodiment, the prosthetic valve can be delivered and implanted via a catheter, by configuring the frame 1405, the horizontal member 1410, and the tarp 1420 to be sufficiently flexible, telescoping, lockable in two different states/positions, compressible, foldable, bendable and or assembled (including any means known to POSA. Any additional features such as arms, skirts, and visualizing, and/or leaflet cutting methods/embodiments discussed in this application or those known to POSA may be used to securely implant this device in the aortic valve.

In an alternate embodiment, the device frame 1405 can have a curved 1425 landing zone for the tarp 1420, configured to improve hemodynamics/hemocompatibility. FIGS. 25F and 25G show states during systole and diastole, respectively.

In an alternate embodiment, the device frame 1405 can have a base with holes 1430, as in FIG. 25H.

In an alternate embodiment, the device frame 1405 can have a base with any configuration or designs of flow pattern 1440, optimized for improved hemodynamics/hemocompatibility, as in FIG. 25I.

In alternate embodiments, the leaflets can be tricuspid or more. The leaflet/Tarp 1420 in FIG. 25 can be single structure or multiple structures assembled together. The leaflet can be attached to the horizontal member 1410 or to the frame 1405 using any known fastening, gluing, welding, press-fitting, riveting, methods. Alternatively, the trap 1420 can be attached directly to the aorta, aortic leaflets. Alternatively, the trap 1420 can be attached directly or indirectly via a connecting feature to the combination or tissue, frame 1405, and/or horizontal member 1410.

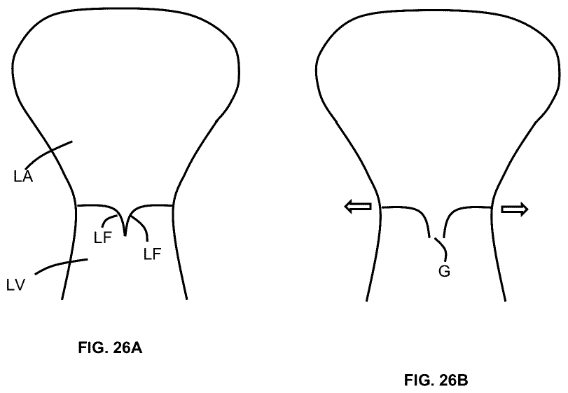

FIG. 26 is an invention for treating valve regurgitation, for example, heart valves such as mitral, tricuspid, aortic, pulmonary. Nevertheless, this idea can be applied to any valve in the body. FIG. 26A shows a normal mitral valve.

FIG. 26B shows a diseased mitral valve with regurgitation caused due to annular dilatation. One exemplary solution/method/device of this invention is to deploy at least one atraumatic ring, just above the annulus. For example, a ring may be placed above (up stream) or below (down stream) the annulus. For example, a spiral ring may be placed over and under the annulus and/or leaflets. Let the tissue fully encapsulate the ring over an extended period of time (typically >30 days) In a follow-up procedure, return to progressively tighten/compress the ring in one or multiple visits, until the gap between the leaflets are reduced or fully closed, to mitigate regurgitation.

In an alternate embodiment, the valve can be configured to auto-cinch over a period to time to a predetermined diameter or shape. This can be achieved by using biodegradable spacers, bio-degradable tube, biodegradable coating, or any other similar means, where in, the ring is held in an expanded state acute and over period of time, shrinks or compresses. One example of the invention is to insert a spring of known diameter, stretch it using a biodegradable tube or spacers, and insert it inside the ring cross-section. Over period of time, the tube or spacer degrades or dissolves and the spring compressed the ring to a pre-determined shape or size. Alternatively, the ring maybe filled with biodegradable material. Alternatively, a balloon maybe used to control the shape and size both acute and over period of time. Alternatively, remotely controlling motors, actuators, wireless, RF, fluidics, thermal, chemical, mechanical means may be used to remotely control the cinching. In an alternate example, material prosperities, such as creep, sublimation, etc can be used.

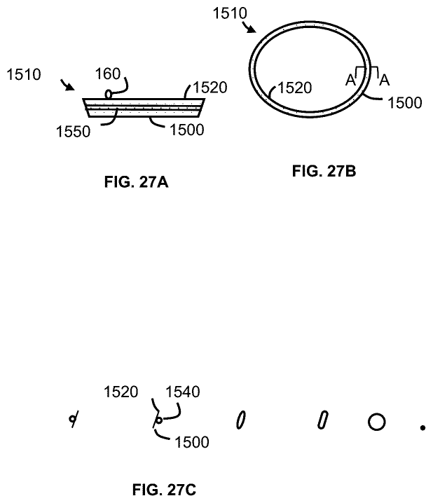

FIG. 27A shows side view of an exemplary ring based annular cinching device. In this exemplary design, a stent design cut from a tube is used and a typical compression spring of circular cross-section is fastened, welded, sutured, or interlaced. A POSA can understand, the ring by passing a string through the compression spring, it can be compressed by cinching the string. While this is an exemplary method, any other method can be used to compress, change shape or size of the ring over period of time.

FIG. 27B shows top view of an exemplary ring based annular cinching device. Alternate embodiments of these can be configured to be of any shape that can be closer to anatomical shapes such as D-shape or any patient specific profile.

FIG. 27C shows various exemplary cross-sectional profiles of the ring. The ring can be made of compression spring with typical circular profile, a stent with a compression spring, a flattened compression spring with an oval, rectangular, triangular, trapezoidal, c-shaped, s-shaped or any other suitable profile. The compressible springs can be made of wire, sheet, tube, or 3D printed. It can have typical stent, braided, woven, coiled, or any suitable means. On the tissue interfacing side, the ring can have miniature barbs or other frictional elements that cause none or minimal trauma to the tissue, both acute (when repeatedly deployed and repositioned) or chronic (over period of time should not cause any ischemia, necrosis, conduction disruptions, etc.). This, unlike current technology, that uses cork screws to fasten deep (>1.5 mm) into the tissue.

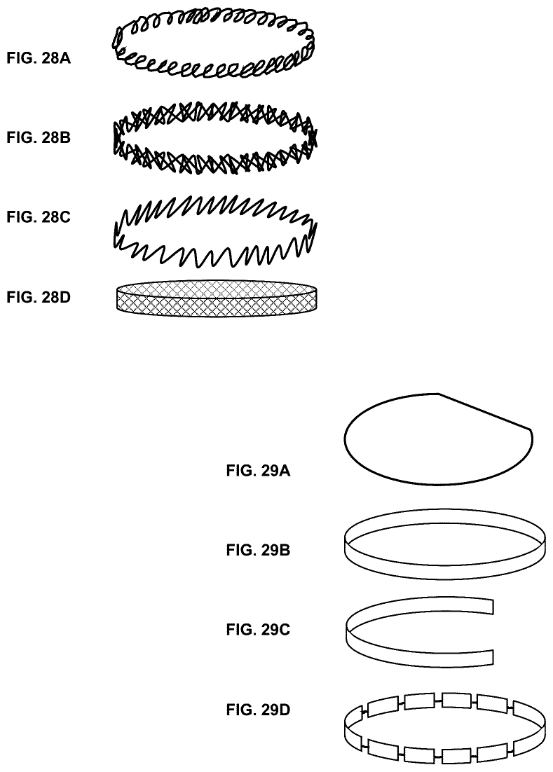

FIG. 28A to FIG. 28D shows various exemplary embodiments of the ring made from wire, sheet, or tube.

FIG. 29 shows various shapes, such as ring, c-shape, d-shape. etc. These can be made of metal, plastic, ceramic, composite, laminar, monolithic, layered, assembled, covered, coated, spun wrapped, molded, over-molded, etc. The frictional elements can be knots, barbs, v, w, s, flat, or rounded edged barbs, rough surface, rough fabric, glue based, screws, barb anchors, or any other means known to POSA to securely attach to tissue, at least until the tissue encapsulation is complete. These frictional elements can be fully atraumatic (non-penetrating the tissue) or penetrating (with minimum trauma). Instead of one continuous structure, the device can be small segments, delinked or linked chain, that can be assembled in the body or at the OR table. Sutures, strings, magnets, pull wires and any suitable technique based on the device segment configuration maybe used to assemble or dissemble, to enable loading inside the catheter. The re-capture, retrieval, re-adjustment features can be radio-active, radio-opaque, echogenic, rf id, or any means to improve visualization, commonly known to POSA.

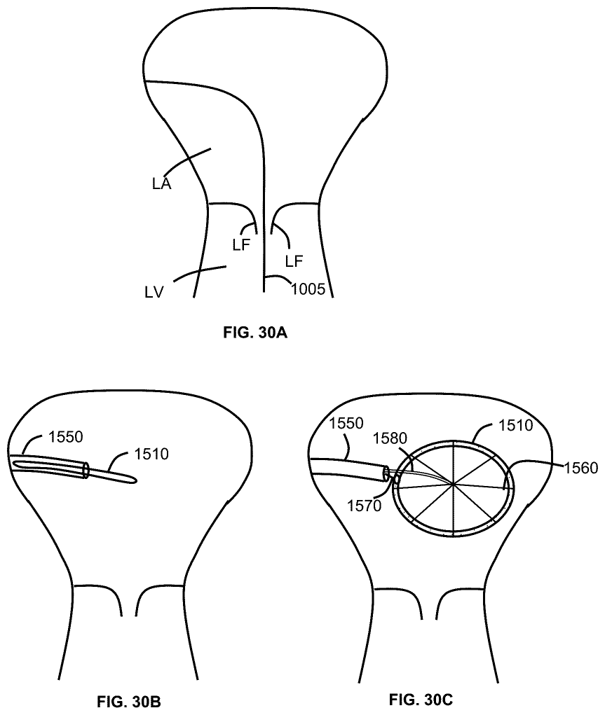

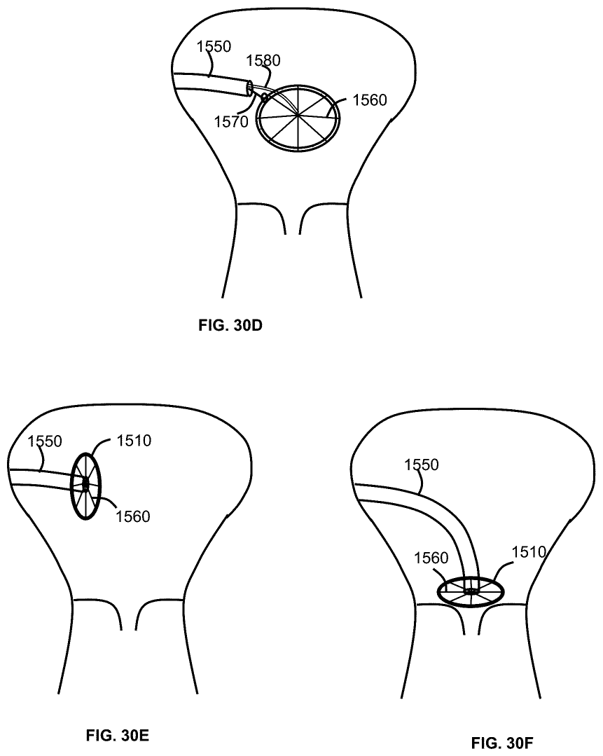

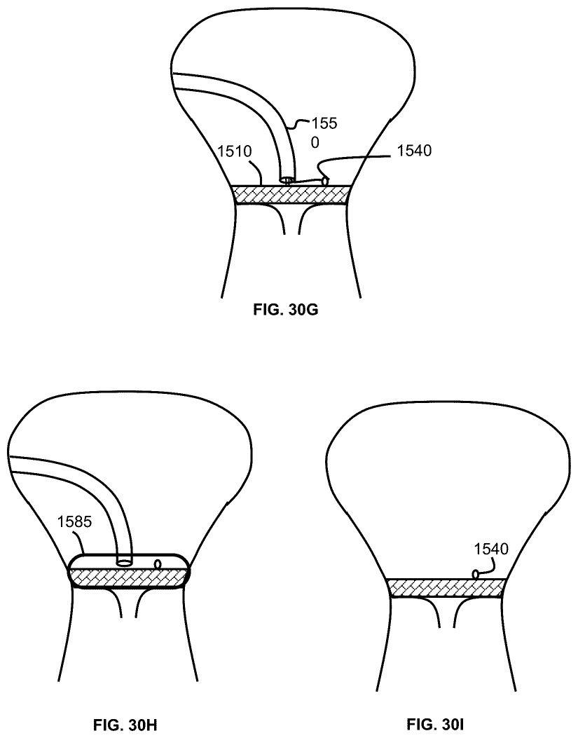

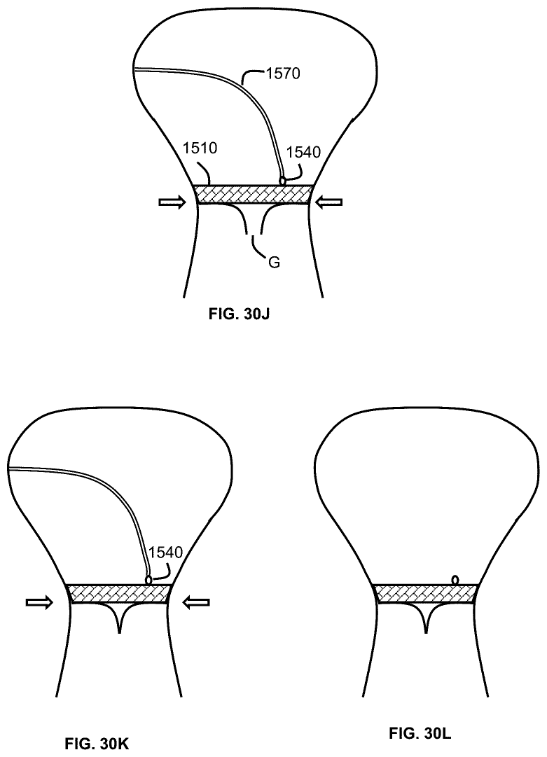

FIG. 30 shows an exemplary method of Orifice repair. FIG. 30A shows a diseased mitral valve with dilated Orifice and a gap G in the mitral leaflets, causing MR. A guidewire is first introduced using transeptal route. Typically, the guidewire is passed via femoral vein access point, up through the Inferior Vena Cava, into the right atrium, and finally into the left atrium on passing through the septa. (Alternatively, left atrium can be accessed from jugular vein. Alternatively, it can be accessed from the femoral artery, aortic valve, left ventricle and then left atrium).

FIG. 30B shows the catheter inserted over the guidewire into the left atrium. The guidewire may optionally be retracted and removed, once the catheter is in place. The ring 1510 is loaded in folded configuration is shown being pushed out. (Note: alternatively, the ring can be compressed or both compressed and folded. Other alternate means can be straightening it in the catheter, in case of a c-shape. Alternatively, any means to load a device, commonly used in catheter anywhere in the body may be used. One advantage of this invention is that it allows for the stent to be folded, flattened, and loaded along the length, thereby, reducing the cross-section of the catheter. Additionally, it can be curved to further reduce the profile of the catheter. Typically, stents are compressed radially/circumferentially, which increases the size of the catheter.

FIG. 30C shows the device 1510 fully out in its expanded state. The device can be detachably held in place using commonly used catheter techniques such as sutures, wires, tubes, etc. For example, the device is being held using detachable radial sutures that pass through tube 1580. The adjustable/retrieval feature 1540 is being connected via torque cable 1570.

FIG. 30D shows the device 1510 in shrunk (compressed) state. This can be achieved either by pulling in the radial sutures 1560 via tube 1580 or by actuating feature 1570. Actuation of feature 1570 can be via turning, rotating, pulling, cinching, inflation/deflation, pneumatic, rf, remote, wireless, electrical, electronic, motorized, geared, toothed, etc.

FIG. 30E shows the device 1510 flush with the catheter 1550. This can be done by retracting the tube 1580.

FIG. 30F shows the device 1510 positioned above the valve by steering the catheter 1550.

FIG. 30G shows the device 1510 in expanded state. The extent of expansion can be controlled either be pre-sizing the device 1510 prior to selecting the size using known catheter techniques such as echocardiography for example. Alternatively, a smart force sensor or size sensor, or surface contact sensor of mechanical, electrical or fluidic, imaging, for example, may be used to ensure proper apposition and placement.

Controlled apposition can be achieved either by actuating 1570 or by pulling in the radial sutures 1560 via tube 1580.

FIG. 30H show alternate and optional method of ensuring apposition by using a balloon, which is inflated to compress the device 1510 against the atrial valve/Orifice. This compression can also be used to securely insert/engage the frictional elements to the annulus/tissue.

FIG. 30I shows the device 1510 implanted. All delivery system has been detached and removed. Optionally, in an alternate embodiment, the actuating feature 1570 can be left attached to the implant and secure transcutaneously or subcutaneously for continued access to adjustment. Optionally, the actuation feature 1570 can be incorporated with sensors to continuously assess the regurgitation. Post sufficient time for robust tissue encapsulation of the device 1510, it can be cinched to reduce the gap between the leaflets, as shown in FIG. 30K. Note, this cinching can be done over a period of time (once a day, week, month or year) or all at once. Alternatively, some controlled cinching can be configured via elastic recoil of the material or other material properties. In an alternate method, the ring 1510 can be configured to be easily cinched but harder to expand or using superelastic materials with hysteresis (different plateaus) or interlocking mechanisms that prefer cinching to expansion.

FIG. 30L shows the final state of the device 1510 with complete treatment or mitigation of the regurgitation. One advantage of this invention is that the device 1510 allows for multimodality treatment, for example, this invention allows or is compatible with edge-to-edge repair and chordal repair. One advantage of this invention is that annulus repair can be done before or after edge-to-edge or chordal repair. One advantage of this invention is that the device 1510 allows for multimodality treatment, for example, this invention allows or is compatible prosthetic valve replacement. One advantage of this invention is that Orifice repair can be done before or after valve replacement. One advantage of this invention is that annular repair forms a scaffolding for the replacement valve.

One advantage of this invention is that it allows for chronic cinching over period of time, preferably after a lag time of 30 days.

One advantage of this invention is that by using outward force (such as a balloon, as described in FIG. 30H), frictional elements (barbs, coils, screws, anchors and or wires) can be securely inserted into the annular tissue, thus allowing for acute cinching and or chronic cinching, without the risk of dislodgement.

One advantage of this invention is that the frictional elements can be pushed in, or twist or rotated to engage with the tissue.

In one exemplary method, the ring 1510 contains minimally penetrating frictional elements is inserted into the left atrium, the ring is then positioned about the annulus under standard fluoroscopy and echo guidance. The ring's frictional elements are anchored into the tissue via a balloon 1585 or by twisting/rotating/pulling/pushing the rings to securely engage or attach it to the annulus. The ring is then deployed, causes one or two step cinching (acute and/or chronic). Acute cinching can be configured via natural elastic recoil of any elastic material when expanded. Chronic cinching can be configured via one-way preferred progressive cinching following the nature expansion and contraction of the beating heart, or via remote controlled cinching or biodegradable controlled cinching.

### General Considerations

Sensors and actuators that may be used in relation to this invention, to improve the safety, ease of use, and efficacy of the delivery system and fixation device. Sensors and actuators may be used to assist and evaluate device delivery (acute) and efficacy (acute or chronic). Sensors and actuators maybe active or passive, removable or implantable and may provide acute or chronic physiological or non-physiological data to assess or evaluate patient health. Sensors and actuators maybe active or passive, removable or implantable and provide acute or chronic physiological or non-physiological data to access or evaluate implant integrity and or function. Sensors may be used for visualization: thermal, optical, ultrasonic (including ICE), OCT, fluoroscopic Sensors and actuators maybe electrical, mechanical, magnetic, RF, chemical or combination. Sensors and actuators maybe wired or wireless and may communicate with mobile or fixed external interface. The catheters of the present invention may be used as a conduit for external sensors, for example pressure sensor replacing Swan-Ganz catheter. The term sensor, electrode, transducer, IC, circuit, chip and actuator may be used interchangeably. Sensors and actuators listed are for examples only. Any suitable metal or polymer or ceramic, organic or inorganic, flexible or rigid, matrix or material and their combinations may be used to produce the desired sensors and actuators. Further, motors may be used to steer the catheters and deploy the device. For example, motors may be used instead of manual knobs or levers to pull or push on the actuation sutures or steerable catheter pullwires or other common mechanisms.

All implant embodiments described in this invention may be optionally covered, wrapped, coated, or the like to improve biocompatibility and tissue interface. Suitable coverings can be fabric, web, fibrous, braid, woven or non-woven. The coatings can be metallic, ceramic, polymeric, or combinations thereof. Suitable metallic coatings include titanium, TiN, tantalum, gold, platinum, and alloys thereof. Suitable ceramic and inorganic coatings include titanium dioxide, hydroxyapatite, CaP, and the like. Suitable polymeric coatings include fluoropolymers, e.g. PTFE, PFA, FEP, ECTFE, ETFE; parylene, polyester, PET, polypropylene, polyurethane, PEEK, PVDF, HDPE, LDPE, UHMWPE, phosphorylcholine, THV, and the like. Suitable biodegradable include poly(lactic acid), poly(glycolic acid), polydioxanone, poly(ε-caprolactone), polyanhydride, poly(ortho ester), copoly(ether-ester), polyamide, polylactone, poly(propylene fumarate), and their combinations. Such metallic, ceramic and/or polymeric coatings are listed as examples only. Any suitable metal, ceramic, polymer, and combination thereof may be used to produce a desirable coating.

In one particular exemplary embodiment of a medical method, a user assesses the regurgitation of valve leaflets through one or more medical imaging methods including, but not limited to fluoroscopy and ultrasound. Based on the assessment of coaptation depth, profile, disease and or size of the leaflets, one or more sizes of straight or curved or a combination shape device is implanted. The advantage of deploying a selected shape and size of implant is to improve efficacy, safety and minimize the number of device implants.

Any of the implant arms disclosed herein may comprise one or more telescoping elements.

For purposes of this description, certain aspects, advantages, and novel features of the embodiments of this disclosure are described herein. The disclosed methods, apparatuses, and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present, or problems be solved.

Although the operations of some of the disclosed methods are described in a particular order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods. As used herein, the terms “a”, “an” and “at least one” encompass one or more of the specified elements. That is, if two of a particular element are present, one of these elements is also present and thus “an” element is present. The terms “a plurality of” and “plural” mean two or more of the specified elements.

For purposes of this description, certain aspects, advantages, and novel features of the embodiments of this disclosure are described herein. The disclosed methods, apparatuses, and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present, or problems be solved.

Although the operations of some of the disclosed methods are described in a particular order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods. As used herein, the terms “a”, “an” and “at least one” encompass one or more of the specified elements. That is, if two of a particular elements are present, one of these elements is also present and thus “an” element is present. The terms “a plurality of” and “plural” mean two or more of the specified elements.

As used herein, the term “and/or” used between the last two of a list of elements means any one or more of the listed elements. For example, the phrase “A, B, and/or C” means “A,” “B,” “C,” “A and B,” “A and C,” “B and C” or “A, B and C.”

As used herein, the term “and/or” used between the last two of a list of elements means any one or more combinations of the listed elements. For example, the phrase “1, 2, 3, . . . 9, and/or 10” means “1”, “2”, “3” and so on until “10”, or between “1 and 2”, “1 and 3”, and so on to between “1 and 10” or between any other combination of the numbers from “1 to 10”, for example, the value can be between 8 and 10.

As used herein, the term “coupled” generally means physically coupled or linked and does not exclude the presence of intermediate elements between the coupled items absent specific contrary language.

Although many embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described above. For all of the embodiments described above, the steps of any methods need not be performed sequentially.

What is claimed is:

1. A method of implanting an aortic heart valve, the method comprising:
a. deploying one or more temporary valves within an aorta of a patient;
b. establishing a blood path to supply coronary arteries of the patient while the temporary valve is in place;
c. implanting a prosthetic valve within the aorta of the patient, the prosthetic valve comprising actuating arms; and
d. actuating the actuating arms of the prosthetic valve to securely grasp aortic leaflets of the patient.

2. The method of claim 1, further comprising:
a. deploying temporary valves above and below the native heart valve;
b. establishing a flow path between the two additional temporary valves, thereby creating a space with no blood flow at the native valve;
c. establishing a continuous optically clear fluid flush within the space; and
d. using an optical camera to visualize the aortic valve region to aid the procedure.

3. The method of claim 2, further comprising:
a. using a heart pump to aid flow between the two temporary valves.

4. The method of claim 1, further comprising:
a. cutting of diseased heart valve leaflets to create space for the prosthetic valve.

5. The method of claim 2, further comprising:
a. cutting of diseased heart valve leaflets to create space for the prosthetic valve.

6. The method of claim 3, further comprising:
a. cutting of diseased heart valve leaflets to create space for the prosthetic valve.

7. The method of claim 1, further comprising:
a. implanting the prosthetic valve at the level of the native aortic leaflets and below the coronary artery ostia.

8. The method of claim 1, further comprising:
a. implanting the prosthetic valve device that comprises of a frame with actuatable arms and a modular insertable and replaceable prosthetic leaflets that can be removed and replaced at a later date post procedure.

9. The method of claim 1, further comprising:
a. implanting a prosthetic valve with a frame of higher compliance, to reduce trauma and retain normal tissue compliance.
